(19) United States
(12) Patent Application Publication
(10) Pub. No.: US 2020/0261239 A1
(43) Pub. Date: Aug. 20, 2020

(54) SPINAL IMPLANT AND METHODS OF USING THE SAME

(71) Applicant: Halo Development LLC, San Antonio, TX (US)

(72) Inventor: Christopher Dennis Chaput, San Antonio, TX (US)

(21) Appl. No.: 16/793,803

(22) Filed: Feb. 18, 2020

Related U.S. Application Data
(60) Provisional application No. 62/806,353, filed on Feb. 15, 2019.

(51) Int. Cl. A61F 2/44; A61F 2/30

(52) U.S. Cl. CPC A61F 2/4425; A61F 2/30907; A61F 2002/30067; A61F 2002/30273; A61F 2002/3092; A61F 2002/30919; A61F 2002/30321

(57) ABSTRACT

Medical implants may include a core body of a first material and one or more attached portions of a second material that enhances osseointegration into the medical implant. The attached portions may include wall-like shapes, sleeve-like shapes, or combinations thereof. The attached portions may be attached to perimeter surfaces of the core body of the implant. The attached portions may conform to and be in substantially continuous contact with the portions of the perimeter surfaces to which they are attached. The attached portions may generally sheathe the geometric area of the portions of the perimeter surfaces to which they are attached.

### CROSS REFERENCE TO RELATED APPLICATION

This application claims the benefit of and priority to U.S. Patent Application No. 62/806,353, filed Feb. 15, 2019, entitled “SPINAL IMPLANT AND METHODS OF USING THE SAME,” which is incorporated herein by reference as if set forth in its entirety.

### BACKGROUND

Bone in-growth to medical implants, also referred to as osseointegration, is a major determinant of implant success. Previous medical implants, such as spinal implants, typically include materials known to be highly biocompatible, such as, for example, polyether ether ketone (PEEK). However, the highly biocompatible property of these implant materials is typically paired with properties that are not conducive to osseointegration. Consequently, surface modifications to PEEK implants are often required to provide an implant surface conducive to osseointegration. For example, previous medical implants formed from PEEK demonstrate poor cellular adhesion, hydrophobicity, and other physical properties that reduce osseointegration. Therefore, the implants require surface modification to promote osseointegration. However, PEEK material may be more challenging to modify by typical methods used for other orthopedic materials, which makes surface modification more costly and complicated.

Previous approaches to overcoming the challenges of surface modification include plasma-spraying titanium onto the surfaces of medical implants to provide a layer that is more easily modified by the typical surface modification methods. However, in such implants, the sprayed on titanium layer may separate from the PEEK portion of the implant (e.g., delamination) due to biomechanical stresses experienced within the human body. Delamination of the titanium layer can lead to complications at the implant site, as well as systemic complications, for example, if delaminated titanium portions or particles migrate from the implant and throughout the body.

Therefore, there is a long-felt, but unmet need for medical implants, and methods for producing the same, that are both highly biocompatible and include surfaces that are modifiable towards the promotion of osseointegration.

### BRIEF SUMMARY OF THE DISCLOSURE

Briefly described, and according to one embodiment, aspects of the present disclosure generally relate to spinal implants and methods of using the same. Spinal implants are typically made from a variety of materials that have advantageous properties. However, each material presents one or more tradeoffs.

As one example, polyether ether ketone (PEEK) is highly biocompatible, has a modulus of elasticity similar to cortical bone, does not generate artifacts on MRI scans, is radiolucent (i.e. does not create artifacts on radiographs), and is relatively inexpensive and generally easy to manufacture. However, surfaces of PEEK implants exhibit poor cellular adhesion, are generally hydrophobic, and have physical properties that reduce (or at least do not promote) osseointegration. Consequently, in previous implants formed from PEEK, surface modification is typically required for improving osseointegration.

As a second example, titanium is highly modifiable at a micro- and nano-surface level, generally interfaces well with bone, and promotes osseointegration. However, titanium has a modulus of elasticity that is much higher than that of bone, obscures bone formation on radiographs, and creates artifacts on MRI scans. In titanium implants, the disparity in moduli between the titanium and bone materials may create microstrain conditions of pathologic overload and cause implant failure.

As discussed herein, a previous approach to producing multi-material implants is to plasma-spray titanium or other materials onto a surface of a PEEK implant to create an implant with a titanium surface, but a PEEK body, potentially combining some advantageous properties of both materials. However, in such implants, the sprayed on titanium layer may separate from the PEEK portion of the implant (e.g., delamination) due to biomechanical stresses experienced within the human body. Delamination of the titanium layer can lead to complications including, but not limited to, reduced osseointegration, aseptic loosening, inflammation in surrounding tissues, for example, if particle debris is created from surface abrasion of the exposed implant, bone resorption, and other negative implant outcomes.

Various aspects of the spinal implants discussed herein include a combination of PEEK and titanium (or other suitable materials), where titanium plates, walls, and/or sleeves are attached to a PEEK body. In particular embodiments, the titanium plates, walls, and/or sleeves, can add a number of advantages to a PEEK implant, particularly, improved surface texture and bone-interface. Further, these titanium plates, walls, and/or sleeves can be attached to a PEEK body through various mechanisms (as discussed herein), which may eliminate potential issues with delamination found with implants with plasma-sprayed titanium layers. The plates, walls, and/or sleeves may be any suitable thickness. In one embodiment, the plates, walls, and/or sleeves includes a thickness of about 0.008 inches. In various embodiments, the plates, walls, and/or sleeves include a thickness between about 0.001-0.0394 inches.

In some embodiments, thicker plates, walls and/or sleeves are implemented to accommodate biomechanical and/or radiographic requirements for the specific implant site. According to one embodiment, the plates, walls, and sleeves of implants described herein include macro-porous features, macro-scale roughness, and nano-scale porous features that promote biologic activities (e.g., osseointegration and wound healing) into and around surfaces of the implants. In at least one embodiment, the enhanced surfaces of the plates, walls, and sleeves of the implants provide a technique for improving osseointegration in PEEK implants that does not require modification to PEEK portions thereof.

According to a first aspect, a spinal implant including: A) a PEEK body including: 1) a body top surface including a top surface outer edge; 2) a body bottom surface including a bottom surface outer edge; and 3) a perimeter surface between the top surface outer edge and the bottom surface outer edge and including four side surfaces; and B) a titanium enhancement material layer that is about 0.001-0.010 inches thick, affixed to the PEEK body, and covering at least two of the four side surfaces of the perimeter surface, wherein the enhancement material layer includes: 1) a layer top surface within the same plane as the body top surface; 2) a layer bottom surface within the same plane as the body bottom surface; and 3) at least one property modification.

According to a second aspect, the spinal implant of the first aspect or any other aspect, wherein the at least one property modification includes increased roughness.

According to a third aspect, the spinal implant of the first aspect or any other aspect, wherein the at least one property modification includes increased porosity.

According to a fourth aspect, the spinal implant of the first aspect or any other aspect, wherein the enhancement material layer includes one or more distinct portions, each distinct portion covering at least a portion of one of the four side surfaces of the perimeter surface.

According to a fifth aspect, the spinal implant of the second aspect or any other aspect, wherein the enhancement material layer covers at least a portion of all four side surfaces of the perimeter surface.

According to a sixth aspect, the spinal implant of the first aspect or any other aspect, wherein the enhancement material layer includes a sleeve covering all four of the four side surfaces of the perimeter surface.

According to a seventh aspect, the spinal implant of the sixth aspect or any other aspect, wherein: A) the PEEK body defines at least one opening; B) the body top surface includes a top surface inner edge; C) the body bottom surface includes a bottom surface inner edge; and D) an inner perimeter surface between the top surface inner edge and the bottom surface inner edge.

According to an eighth aspect, the spinal implant of the seventh aspect or any other aspect, wherein: A) the spinal implant includes a second enhancement material layer affixed to the PEEK body, covering at least a portion of the inner perimeter surface, and including: 1) a second layer top surface within the same plane as the body top surface; and 2) a second layer bottom surface with the same plane as the body bottom surface.

According to a ninth aspect, the spinal implant of the first aspect or any other aspect, wherein the enhancement material layer defines one or more openings through the enhancement material layer.

According to a tenth aspect, the spinal implant of the ninth aspect or any other aspect, wherein the one or more openings include encoded information.

According to an eleventh aspect, the spinal implant of the tenth aspect or any other aspect, wherein the encoded information is visible via a radiograph.

According to a twelfth aspect, the spinal implant of the first aspect or any other aspect, wherein the PEEK body includes a shape selected from the group of: prisms, pyramids, solids of revolution.

According to a thirteenth aspect, a spinal implant including: A) a PEEK body defining at least one opening and including: 1) a body top surface including a top surface outer edge; 2) a body bottom surface including a bottom surface outer edge; and 3) a perimeter surface between the top surface outer edge and the bottom surface outer edge; and B) an enhancement material layer that is about 0.001-0.0394 inches thick, affixed to the PEEK body, and covering at least a portion of the perimeter surface, wherein the enhancement material layer includes: 1) a layer top surface within the same plane as the body top surface; and 2) a layer bottom surface within the same plane as the body bottom surface.

According to a fourteenth aspect, the spinal implant of the thirteenth aspect or any other aspect, wherein the enhancement material layer includes titanium.

According to a fifteenth aspect, the spinal implant of the fourteenth aspect or any other aspect, wherein the perimeter surface includes four side surfaces.

According to a sixteenth aspect, the spinal implant of the fifteenth aspect or any other aspect, wherein the enhancement material layer includes at least two portions, each portion covering one of the four side surfaces.

According to a seventeenth aspect, the spinal implant of the sixteenth aspect or any other aspect, wherein the enhancement material layer includes four portions, each portion covering one of the four side surfaces.

According to an eighteenth aspect, the spinal implant of the seventeenth aspect or any other aspect, wherein the enhancement material layer includes a sleeve covering all four of the four side surfaces.

According to a nineteenth aspect, the spinal implant of the eighteenth aspect or any other aspect, wherein: A) the body top surface includes a top surface inner edge; B) the body bottom surface includes a bottom surface inner edge; and C) an inner perimeter surface between the top surface inner edge and the bottom surface inner edge.

According to a twentieth aspect, the spinal implant of the seventeenth aspect or any other aspect, wherein: A) the spinal implant includes a second enhancement material layer affixed to the PEEK body, covering at least a portion of the inner perimeter surface, and including: 1) a second layer top surface within the same plane as the body top surface; and 2) a second layer bottom surface with the same plane as the body bottom surface.

These and other aspects, features, and benefits of the claimed medical implants and methods of using the same will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings illustrate one or more embodiments and/or aspects of the disclosure and, together with the written description, serve to explain the principles of the disclosure. Wherever possible, the same reference numbers (if any) are used throughout the drawings to refer to the same or like elements of an embodiment, and wherein:

FIG. 1 shows an exploded view of an exemplary implant according to one embodiment of the present disclosure.

FIG. 2 shows a perspective view of an exemplary implant according to one embodiment of the present disclosure.

FIG. 3 shows a top view of an exemplary implant according to one embodiment of the present disclosure.

FIG. 4 shows a bottom view of an exemplary implant according to one embodiment of the present disclosure.

FIG. 5 shows a front view of an exemplary implant according to one embodiment of the present disclosure.

FIG. 6 shows a side view of an exemplary implant according to one embodiment of the present disclosure.

FIG. 7 shows a side view of an exemplary implant according to one embodiment of the present disclosure.

FIG. 8 shows a back view of an exemplary implant according to one embodiment of the present disclosure.

FIG. 9 shows an exploded view of an exemplary implant according to one embodiment of the present disclosure.

FIG. 10 shows a perspective view of an exemplary implant according to one embodiment of the present disclosure.

FIG. 11 shows a top view of an exemplary implant according to one embodiment of the present disclosure.

FIG. 12 shows a bottom view of an exemplary implant according to one embodiment of the present disclosure.

FIG. 13 shows a front view of an exemplary implant according to one embodiment of the present disclosure.

FIG. 14 shows a side view of an exemplary implant according to one embodiment of the present disclosure.

FIG. 15 shows a side view of an exemplary implant according to one embodiment of the present disclosure.

FIG. 16 shows a back view of an exemplary implant according to one embodiment of the present disclosure.

FIG. 17 shows an exploded view of an exemplary implant according to one embodiment of the present disclosure.

FIG. 18 shows a perspective view of an exemplary implant according to one embodiment of the present disclosure.

FIG. 19 shows a top view of an exemplary implant according to one embodiment of the present disclosure.

FIG. 20 shows a bottom view of an exemplary implant according to one embodiment of the present disclosure.

FIG. 21 shows a front view of an exemplary implant according to one embodiment of the present disclosure.

FIG. 22 shows a side view of an exemplary implant according to one embodiment of the present disclosure.

FIG. 23 shows a side view of an exemplary implant according to one embodiment of the present disclosure.

FIG. 24 shows a back view of an exemplary implant according to one embodiment of the present disclosure.

FIG. 25 shows an exemplary implant with encoded information according to one embodiment of the present disclosure.

FIG. 26 shows an exemplary implant with encoded information according to one embodiment of the present disclosure.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

### Overview

Various aspects of the spinal implants discussed herein include a combination of PEEK and an enhancement material (such as titanium). In various embodiments, “enhancement material” generally refers to a material, the surface of which is modified to promote osseointegration into a core body to which the material is attached. According to one embodiment, walls and/or sleeves including the enhancement material are attached to a central core of material, such as PEEK. In various embodiments, a PEEK core of an implant may be constructed with an interior cavity for allowing bone infill. The embodiments discussed herein are not limited to PEEK and titanium enhancement materials, but contemplate any suitable materials.

In one or more embodiments, a layer of an enhancement material, such as titanium, may be attached to a PEEK core to provide a surface texture (micro or nano) to the PEEK core and to help promote osseointegration of the implant. As would be understood by an individual of ordinary skill in the art, “osseointegration” generally refers to bone growth into an implant. Measurements to holistically assess bone in-growth potential and viability include, but are not limited to, bone-to-implant contact (BIC) value, torque removal force, and crestal bone loss (CBL).

In at least one embodiment, an enhancement material includes one or more substances. In various embodiments, the one or more substances are selected using a plurality of criteria including, but not limited to: 1) biocompatibility; 2) mechanical performance; 3) conductivity; 4) corrosion resistance; 5) reactivity; and 6) osseointegration performance. According to one embodiment, the one or more substances include, but are not limited to, titanium, titanium alloys such as nitinol, silicon nitride ceramic, titanium nitride, and zirconia, or any suitable biocompatible material such that an that appropriate osseoinductive surface and/or sub-surface texture can be created. Implants and core bodies thereof discussed herein are not limited to PEEK materials. In various embodiments, a core body may include one or more materials including, but not limited to: 1) PEEK; 2) polyetherketone (PEK); 3) polyetherketoneketone (PEKK); 4) polyaryletherketone (PAEK); 5) polyetherketoneetherketoneketone (PEKEKK) (e.g., with pyrolytic carbon fibers); and 6) any suitable biocompatible material that has similar modulus of elasticity to cortical bone and is capable of withstanding biomechanical loading.

As will be understood from discussions herein, various techniques may be used to create the present implants. In some embodiments, an enhancement material layer is formed into one or more wall-like shapes that are attached (e.g., affixed or adhered) to a core body of an implant. In various embodiments, the wall-like shapes are referred to as “walls.” In one or more embodiments, a wall may be attached to exterior and/or interior surfaces of a core body. In one example, a core body includes four portions arranged into a generally quadrilateral shape, each of the four portions includes an external surface, and at least one wall is attached to and sheathes a substantial geometric area of each of the four external surfaces.

According to one embodiment, a wall is attached to an exterior surface of a core body in a manner such that the wall is in conformed and substantially continuous contact with the exterior surface of the core body. In other words, in at least one embodiment, walls described herein are formed into shapes that conform to shapes of external surfaces to which the walls are attached. In one example, an external surface includes a width X, a height Y, and a curvature Z. In the same example, a wall attached to the external surface includes a width substantially similar to, but no greater than, the width X, includes a height substantially similar to, but no greater than, the height Y, and includes a curvature substantially similar to the curvature Z.

In some embodiments, a wall sheathes a partial geometric area of a core body surface. In other embodiments, a wall sheathes substantially all of the geometric area of a core body surface. In at least one embodiment, multiple walls are attached to a core body surface. In various embodiments, multiple walls attached to a core body surface may include a predetermined spacing between an edge of one wall and an adjacent edge of another wall.

According to one embodiment, walls are attached to one or more exterior surfaces of a perimeter surface of a core body. In one or more embodiments, a perimeter surface of a core body generally refers to exterior surfaces (including exterior surfaces of interior cavities described herein). In at least one embodiment, the perimeter surface of a core body excludes a top surface and a bottom surface of the core body.

In at least one embodiment, the enhancement material is formed into one or more sleeve shapes that are attached to the perimeter surface of a core body. In other words, in various embodiments, one or more sleeve shapes are attached to an exterior surface (or a portion thereof) of a core body of an implant and/or to an exterior surface of an interior cavity of the core body. In various embodiments, a sleeve shape attached to a core body exterior portion of a perimeter surface is referred to as an “outer sleeve,” and a sleeve shape attached to an exterior portion a perimeter surface is referred to as an “inner sleeve.” In one or more embodiments, an outer sleeve is attached to a core body in a manner such that an interior surface of the outer sleeve is in conformed and substantially continuous contact with the perimeter surface of the core body, and such that the outer sleeve generally sheathes the geometric area of the exterior surface. According to one embodiment, an inner sleeve is attached to a core body in a manner such that an exterior surface of the inner sleeve is in conformed and substantially continuous contact with the perimeter surface defined by an interior cavity of the core body, and such that the inner sleeve generally sheathes the geometric area of the perimeter surface. In at least one embodiment, the walls, inner sleeves, and/or outer sleeves are attached to the core body at the perimeter surface, a top surface, and/or a bottom surface thereof.

It will be understood by an individual of ordinary skill in the art that the walls, outer sleeves, and/or inner sleeves described herein may be dimensioned to substantially conform to dimensions of portions of a core body to which the walls, outer sleeves, and/or inner sleeves are attached. According to one embodiment, the walls, outer sleeves, and/or inner sleeves are dimensioned to sheathe a predetermined portion of a target site. In one example, an external surface includes a width X, a height Y, and a curvature Z. In the same example, a wall attached to the external surface includes a width less than the width X, includes a height less than the height Y, and includes a curvature substantially similar to the curvature Z. Thus, in the same example, the attached wall conforms to the curvature Z of the external surface, but sheathes only a portion thereof.

In at least one embodiment, sleeves and walls described herein are attached to a core body via a variety of methods including, but not limited to: 1) fasteners; 2) snap fitting; 3) press fitting, such as tapered press fit or otherwise; 4) adhesives, for example, selected from groups such as cyanoacrylates, acrylics, epoxies, urethanes, elastomers, and silicones, or from a combination of adhesive groups; and 5) friction lap welding. In various embodiments, an implant includes at least one wall or sleeve secured by press fit or taper locked to the core body of the implant. According to one embodiment, the press fit is implemented using one or more ISO fit standards to achieve secure interference of wall or sleeve surfaces and corresponding surfaces of the core body. In at least one embodiment, the taper lock is implemented using one or more taper lock standards (for example, Morse taper) to achieve secure interference of wall or sleeve surfaces and corresponding surfaces of the core body.

In particular embodiments, the enhancement materials are produced by one or more production modes or additive manufacturing practices (or other manufacturing techniques). In at least one embodiment, the walls, outer sleeves, and inner sleeves described herein include the enhancement materials. According to one embodiment, the walls, outer sleeves, and/or inner sleeves include surface and/or sub-surface topography modification techniques to increase ossification into the enhancement material or implant and/or to generate identifiable structures on the material. Material forming and modification techniques may produce a more porous surface and/or sub-surface finish, thus permitting a more rapid and greater proportion of bone integration into the implant. In one embodiment, an appropriate minimum level of surface porosity of the enhancement material may be 60%. In at least one embodiment, the surface and/or sub-surface of enhancement material (e.g., shapes, such as portions, walls, sleeves, etc., formed therefrom) is modified to demonstrate an increased porosity (e.g., as compared to an unmodified embodiment thereof). Methods of altering and/or forming structures or pores on enhancement material surface and/or sub-surface may include, but are not limited to: 1) etching; 2) particle blasting; 3) micromachining by electrical techniques, electrochemical techniques, and otherwise; 4) coating; 5) selective laser melting (SLM); and 6) selective laser sintering (SLS). According to one embodiment, pores formed on enhancement material surfaces and/or sub-surfaces include a generally circular shape. In one or more embodiments, the pores include a diameter between about 1-100 nm, about 1-10 nm, about 10-20 nm, about 20-30 nm, about 30-40 nm, about 40-50 nm, about 50-60 nm, about 60-70 nm, about 70-80 nm, about 80-90 nm, or about 90-100 nm. In at least one embodiment, structures formed for increasing roughness of enhancement material surfaces and/or sub-surfaces include a length, width, and depth, each measuring between about 1-100 nm, about 1-10 nm, about 10-20 nm, about 20-30 nm, about 30-40 nm, about 40-50 nm, about 50-60 nm, about 60-70 nm, about 70-80 nm, about 80-90 nm, or about 90-100 nm.

In at least one embodiment, the enhancement material (e.g., such as portions, walls, sleeves, etc., formed therefrom) are modified to demonstrate an increased roughness (e.g., as compared to an unmodified embodiment thereof). Methods of increasing roughness enhancement material surfaces and/or sub-surfaces may include, but are not limited to: 1) etching; 2) particle blasting; 3) micromachining by electrical techniques, electrochemical techniques, and otherwise; 4) coating; 5) selective laser melting (SLM); and 6) selective laser sintering (SLS).

In particular embodiments, a wall, outer sleeve, or inner sleeve including an enhancement material is modified to encode logistical data. Data encoding via material modification may be enabled as result of the contrasting radiopaque properties of the enhancement material against the radiolucent properties of the core material. For example, an implant containing a radiolucent core material and a radiopaque outer enhancement material may permit discernment of specific surface features in images generated by imaging modalities.

In various embodiments, information-encoding surface features may be observed via a plurality of imaging methods including, but not limited to: 1) X-ray; 2) magnetic resonance imaging (MRI); 3) multidetector-row computed tomography (MDCT); and 4) computed tomography (CT). According to one embodiment, encoded information includes one or more data elements including, but not limited to, unique device identifiers (UDI), manufacturing and production tracking numbers, patient-specific information, implant-specific information, and procedure-specific information. In one or more embodiments, a wall or sleeve is encoded with information via one or more material subtractive techniques including, but not limited to, etching and micromachining by electrical techniques, electrochemical techniques, or other micromachining techniques. In various embodiments, the information is encoded in one or more formats including, but not limited to: 1) raw text; 2) pore matrices; 3) QR codes; 4) bar codes; and 5) geometric patterns.

In at least one embodiment, the implants discussed herein (e.g., sleeves and/or walls thereof) include a surface coating to achieve desirable surface features and/or implant properties to promote osseointegration. In various embodiments, the implants include a coating of one or more materials including, but not limited to, hydroxyapatite, calcium phosphate, bioactive glass (e.g., bioactive silicates), and/or tricalcium phosphate. In one embodiment, an implant may be coated by one or more techniques including, but not limited to: 1) chemical vapor deposition; 2) electrophoretic deposition; 3) electrochemical deposition; and 4) plasma-enhanced metalorganic chemical vapor deposition. According to one embodiment, the coating includes a thickness measuring between about 20-35 μm, about 20-25 μm, about 25-30 μm, or about 30-35 μm. In one or more embodiments, the thickness of the coating is selected to be sufficiently small such that, following application, the coating does not obstruct a porous and/or rough texture of the applied to wall, sleeve, plate, and/or layer of enhancement material.

As will be understood, the features discussed herein are applicable to any suitable size or shape implant, including, but not limited to: 1) cervical implants; 2) lumbar implants; 3) wedges and/or spacers used between bones (e.g., bones in the spine, bones in the foot and ankle, etc.); 4) buttress implants; 5) stemmed joint and/or hemiarthroplasty implants; 6) hammer toe correction implants; 7) PEEK anchors; 8) craniofacial implants; 9) dental implants; 10) implantable rods; and 11) other interbody fusion implants. Further, the features discussed herein are applicable to various types of medical devices and are not limited to spinal implants. For example, some types of hip replacement devices may be made from PEEK with wall/sleeve features. As such, portions of a hip implant may include enhancement materials discussed herein in the form of walls, plates, sleeves, etc., and may further include encoded information as discussed herein.

### EXEMPLARY EMBODIMENTS

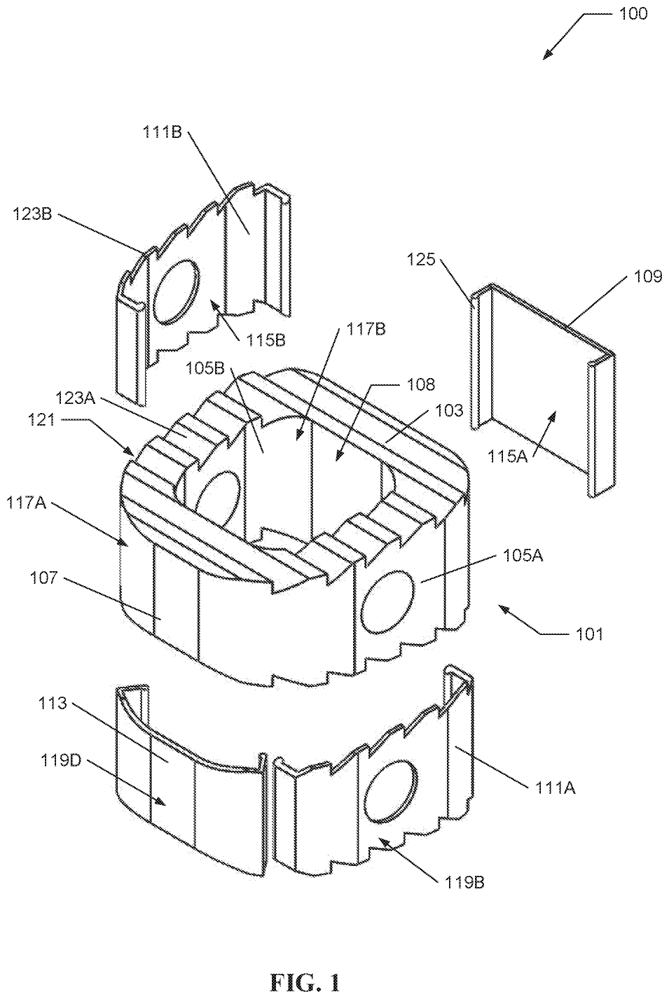

Referring now to the figures, for the purposes of example and explanation of the fundamental processes and components of the disclosed systems and methods, reference is made to FIGS. 1-26, which illustrate exemplary implants according to one or more embodiments of the present disclosure. As will be understood and appreciated, the exemplary, implants shown in FIGS. 1-26 represent merely one approach or embodiment, and other aspects are used according to various embodiments of the present disclosure.

FIG. 1 shows an exploded view of an exemplary implant 100. In at least one embodiment, the implant 100 is an exemplary cervical cage implant. According to one embodiment, the implant 100 includes a core body 101 and one or more walls. In at least one embodiment, the one or more walls include a front wall 109, a first side wall 111A, a second side wall 111B, and a back wall 113.

As will be understood by an individual of ordinary skill in the art, the core body 101 shown in FIG. 1 is non-limiting, and other embodiments of the core body are contemplated herein. In various embodiments, a core body includes one or more shapes including, but not limited to, prisms, pyramids, solids of revolution, ovoids, and other shapes. In one or more embodiments, a core body is shaped substantially similar to one or more of a variety of implants including, but not limited to: 1) cervical implants (e.g., as depicted in the core body 101 of FIG. 1); 2) lumbar implants; 3) thoracic implants; 4) wedges and/or spacers used between bones; 5) buttress implants; 6) stemmed joint and/or hemiarthroplasty implants; 7) hammer toe correction implants; 8) anchors, such as, for example, PEEK anchors; 9) craniofacial implants; 10) dental implants; and 11) implantable rods.

In one or more embodiments, the core body 101 includes a front portion 103 that is integrally formed with a first side portion 105A and a second side portion 105B, and a back portion 107 (e.g., opposite the front portion 103) that is integrally formed with the first side portion 105A and the second side portion 105B. According to one embodiment, the core body 101 includes an interior cavity 108 formed by the integrally formed portions of the core body 101. In at least one embodiment, the interior cavity 108 allows for increased bone growth and infill into the implant 100.

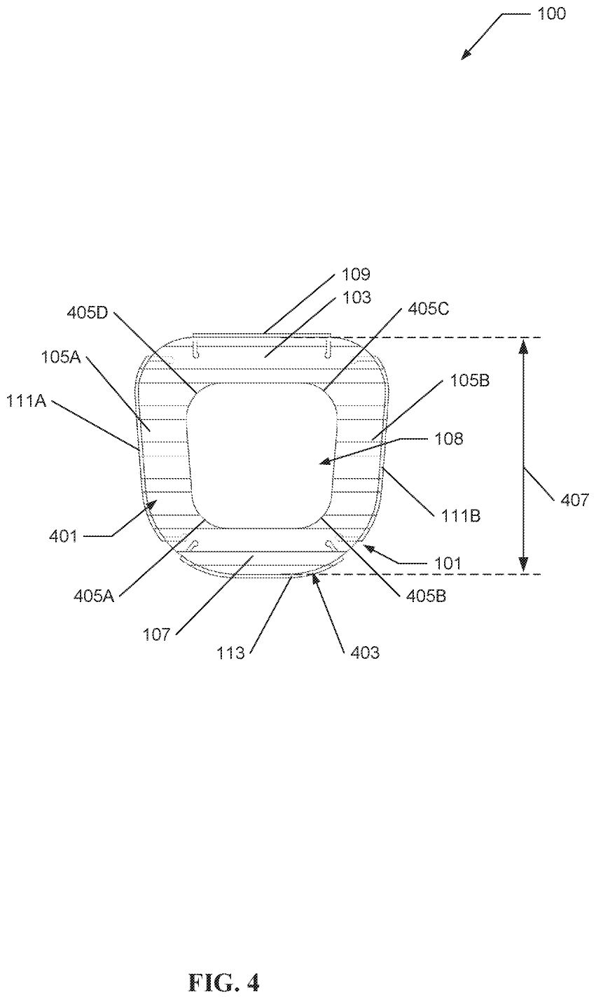

According to one embodiment, each of the walls 109, 111A, 111B, and 113 includes an interior surface 115A or 115B. In various embodiments, upon attachment, the interior surface 115A or 115B of each wall 109, 111A (interior surface not shown), 111B, and/or 113 (interior surface not shown) interfaces with the corresponding portion of the core body 101. In at least one embodiment, the core body 101 includes a perimeter surface 117A, 117B, a top surface 121, and a bottom surface 401 (shown in FIG. 4). In one or more embodiments, the interior surface 115A or 115B of each wall 109, 111A, 111B, and/or 113 attached to the core body 101 is in conformed and substantially continuous contact with the perimeter surface 117A and/or perimeter surface 117B.

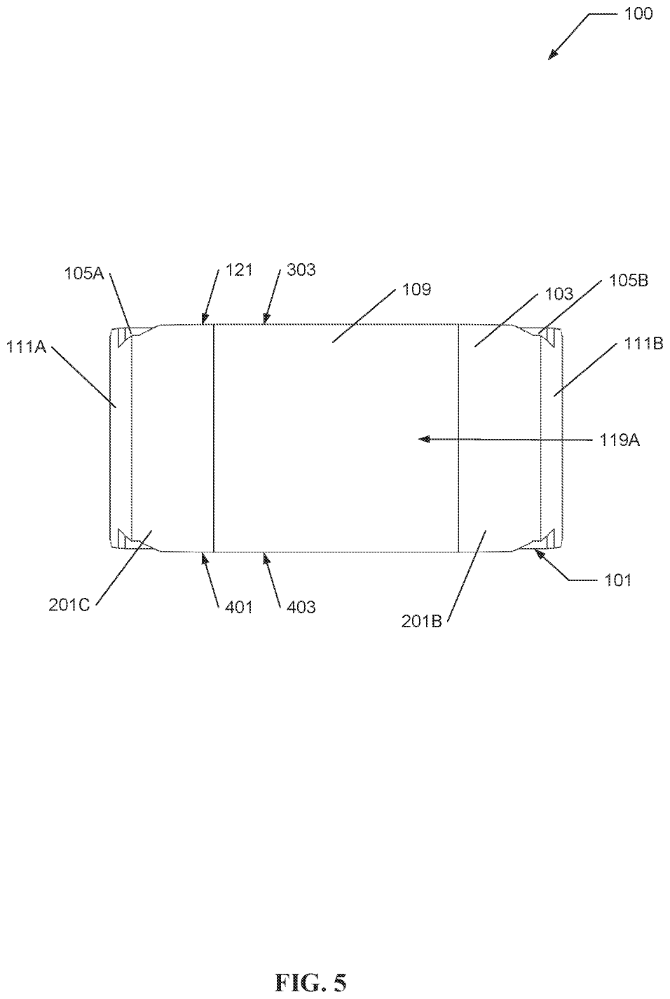

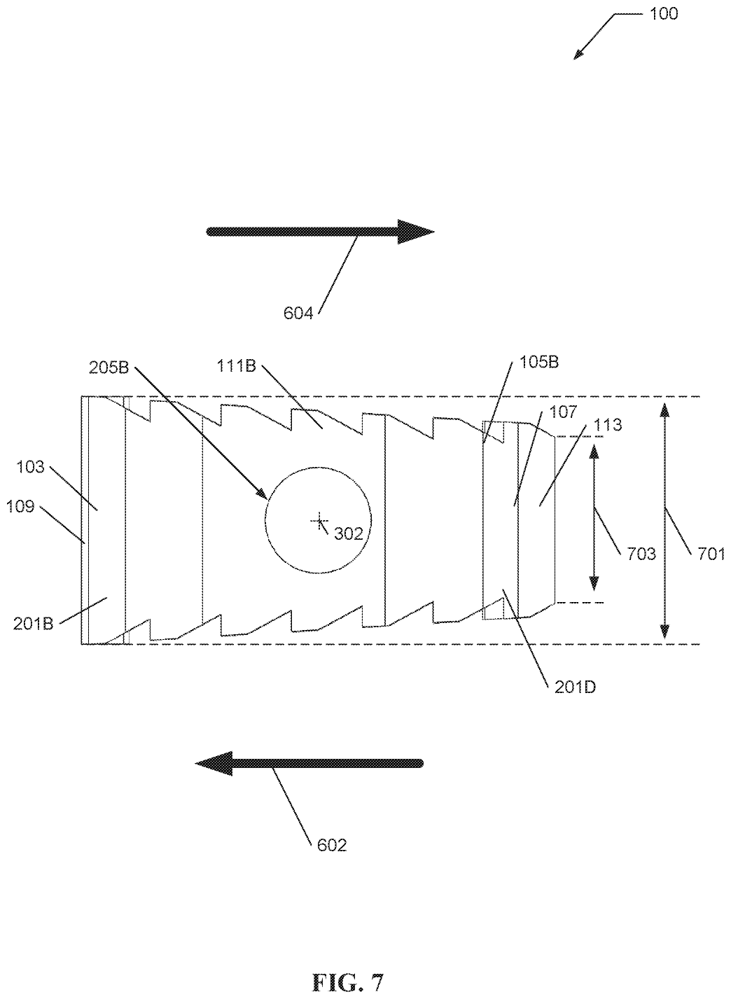

In various embodiments, the walls 109, 111A, 111B, and/or 113 include one or more enhancement materials described herein. In at least one embodiment: 1) one or more front walls 109 are configured (e.g., sized and shaped) for attachment to the front portion 103; 2) one or more first side walls 111A are configured for attachment to the first side portion 105A); 3) one or more second side walls 111B are configured for attachment to the second side portion 105B; and 4) one or more back walls 113 are configured for attachment to the back portion 107. According to one embodiment the walls 109, 111A, 111B, and/or 113 include the interior surface 115A or 115B and an exterior surface 119A (FIG. 5), 119B, 119C (FIG. 7), or 119D. In one or more embodiments, the interior surface 115A or 115B of each wall 109, 111A, 111B, and/or 113 of the implant 100 is formed to conform to a shape of the corresponding perimeter surface 117A, 117B with which the interior surface 115A or 115B interfaces. In one example, a perimeter surface 117A of a front portion 103 includes a particular curved shape. In the same example, an interior surface 115A of a front wall 109 attached to the front portion 103 includes a curved shape substantially similar to the particular curved shape of the perimeter surface 117A, thereby permitting conformed and substantially continuous contact therebetween.

In various embodiments, the interior surface 115A or 115B, the exterior surface 119A, 119B, 119C, and/or 119D, and/or internal portions of the walls 109, 111A, 111B, and/or 113 include one or more property modifications described herein for increasing ossification into the wall 109, 111A, 111B, and/or 113 and/or core body 101. In one example, the exterior surface 119A includes a plurality of porous structures permitting increased and more rapid osseointegration. In one or more embodiments, the exterior surface 119A, 119B, 119C, 119D demonstrates a porosity of at least about 60%, or between about 60-65%, about 65-70%, about 70-75%, about 75-80%, about 80-85%, about 85-90%, about 90-95%, or about 95-99%.

In various embodiments, pores formed into the exterior surface 119A (and/or other surfaces of the wall 109, 111A, 111B, and/or 113) include a width measuring between about 1-700 μm, about 250-300 μm, about 1-50 μm, about 50-100 μm, about 100-150 μm, about 150-200 μm, about 200-250 μm, about 250-300 μm, about 300, about 300-350 μm, about 350-400 μm, about 400-450 μm, about 450-500 μm, about 500-550 μm, about 550-600 μm, about 600-650 μm, about 650-700 μm, about 700 μm, about 700-750 μm. In at least one embodiment, a pore width between about 1-100 μm is selected, for example, to promote adhesion of osteoblasts to the implant 100.

In at least one embodiment, the interior surface 115A or 115B, the exterior surface 119A, 119B, 119C, and/or 119D, and/or internal portions of the walls 109, 111A, 111B, and/or 113 are modified to demonstrate an increased porosity and/or an increased roughness (e.g., as compared to an unmodified embodiment thereof). Methods for increasing roughness and porosity of enhancement material surfaces and sub-surfaces, and for forming structures or pores on the interior surface 115A or 115B, the exterior surface 119A, 119B, 119C, and/or 119D, and/or internal portions of the walls 109, 111A, 111B, and/or 113 may include, but are not limited to: 1) etching; 2) particle blasting; 3) micromachining by electrical techniques, electrochemical techniques, and otherwise; 4) coating; 5) selective laser melting (SLM); and 6) selective laser sintering (SLS).

In at least one embodiment, the core body 101 and/or walls 109, 111A, 111B, and/or 113 include one or more expulsion resistance features 123A, 123B that increase a minimum pull-out and/or pull-through force required to dislodge the implant 100 from a target site. In one example, the one or more expulsion resistance features 123A, 123B include a plurality of ridges arranged and oriented to increase the pull-out forces required to dislodge the implant 100 from a target site following implantation.

As will be understood by an individual of ordinary skill in the art, the one or more expulsion resistance features 123A, 123B are not limited to ridge structures, but may include any suitable structure for reducing a risk of the implant 100 becoming dislodged or expelled from a target site. In at least one embodiment, one or more walls 109, 111A, 111B, and/or 113 are attached to and are in conformed and substantially continuous contact with (e.g., and generally sheath a surface area of) the one or more expulsion resistance features 123A, 123B.

In at least one embodiment, the walls 109, 111A, 111B, and/or 113 include one or more fixation structures 125 that penetrate the core body 101 to attach and secure the walls 109, 111A, 111B, and/or 113 to the core body 101. In at least one embodiment, a fixation structure 125 includes a generally wedge-shaped and/or rounded portion that increases a pullout force required to dislodge the attached wall 109, 111A, 111B, or 113 from the core body 101. In at least one embodiment, top and/or bottom portions of the one or more fixation structures 125 protrude from the corresponding portions of the core body 101 penetrated thereby. According to alternate embodiments, the one or more fixation structures 125 are wholly sub-surface (e.g., the top and/or bottom portions thereof do not protrude from the core body 101).

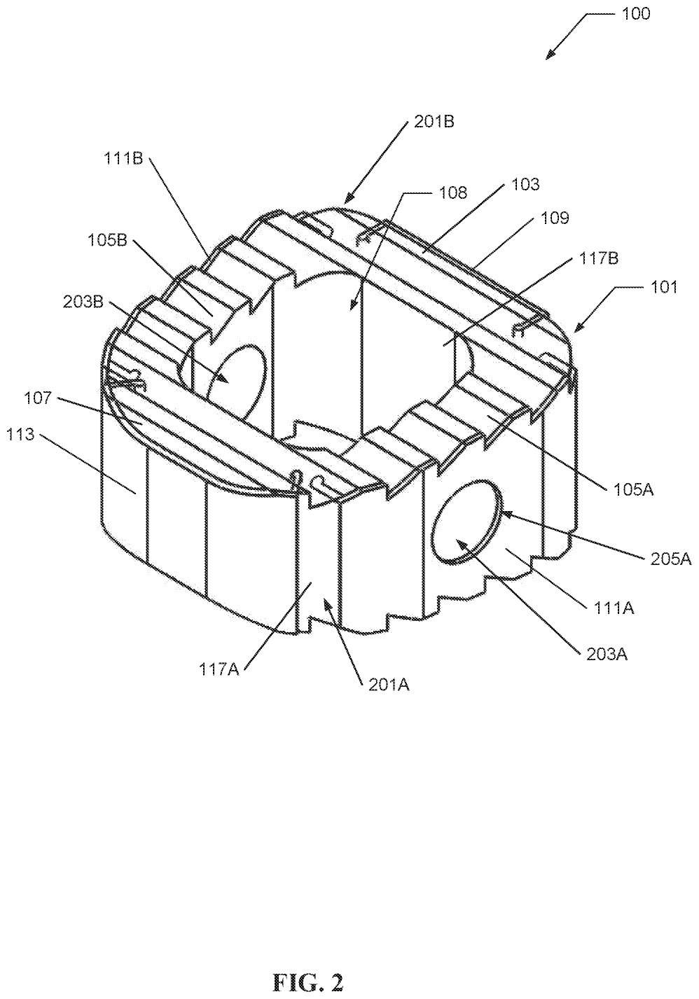

FIG. 2 shows a perspective view of the implant 100. As described herein, in at least one embodiment, the walls 109, 111A, 111B, and 113 are attached to the core body 101. In various embodiments, the walls 109, 111A, 111B, and 113 are attached to the core body 101 in a manner such that: 1) front wall 109 is attached to, is in conformed and substantially continuous contact with, and generally sheathes the perimeter surface 117A (not shown) of the front portion 103; 2) the first side wall 111A is attached to, is in conformed and substantially continuous contact with, and generally sheathes the perimeter surface 117A of the first side portion 105A; 3) the second side wall 111B is attached to, is in conformed and substantially continuous contact with, and generally sheathes the perimeter surface 117A of the second side portion 105B; and 4) the back wall 113 is attached to, is in conformed and substantially continuous contact with, and generally sheathes the perimeter surface 117A of the back portion 107.

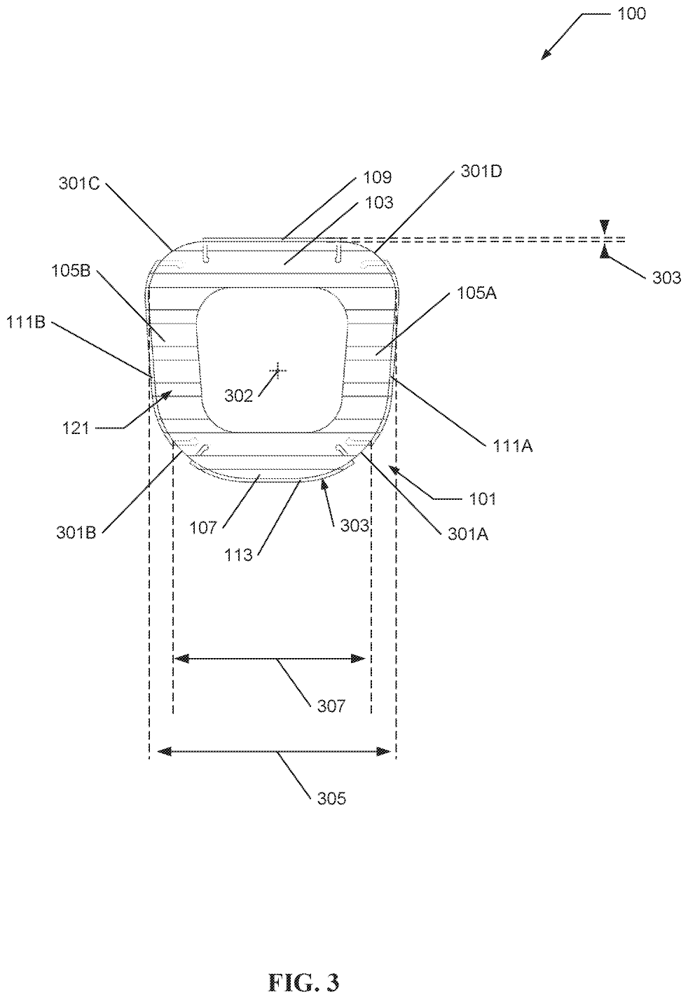

In one or more embodiments, the shape of each wall 109, 111A, 111B, and/or 113 is specific to its intended attachment location on the implant 100. According to one embodiment, the specific geometry and dimensions of a target portion of a perimeter surface 117 forms a template for the specific geometry and dimensions of a corresponding wall 109, 111A, 111B, or 113 that is attached to and in contact with the target portion. In at least one embodiment, the attached walls 109, 111A, 111B, and/or 113 terminate at the top surface 121 and the bottom surface 401 (FIG. 4). In other words, in at least one embodiment, a top surface 303 (FIG. 3) of each wall 109, 111A, 111B, and/or 113 is generally coplanar with the top surface 121 of corresponding portions of the core body 101, and a bottom surface 403 (FIG. 4) of each wall 109, 111A, 111B, and/or 113 is generally coplanar with the bottom surface 401 of corresponding portions of the core body 101. In various embodiments, the wall 109, 111A, 111B, and/or 113 are shaped such that the top surface 303 and bottom surface 403 thereof conform to angles of corresponding front portions 103, side portions 105A, 105B, and back portions 107 of the core body 101.

In one example, the implant 100 is a spinal implant and includes a core body 101 with front portions 103, side portions 105A, 105B, and back portions 107 shaped according to a predetermined lordotic angle. In the same example, corresponding walls 109, 111A, 111B, 113 attached to the front portions 103, side portions 105A, 105B, and back portions 107 are shaped to conform thereto and according to the predetermined lordotic angle.

In a second example, a wedge implant includes a core body shaped according to predetermined osteotomy angles. In the same example, corresponding walls of enhancement material attached to the core body are shaped to conform thereto and according to the predetermined osteotomy angles.

In one or more embodiments, the walls 109, 111A, 111B, and/or 113 do not entirely sheath the perimeter surface 117A. According to one embodiment, the walls 109, 111A, 111B, and/or 113 are sized such that, upon attachment to the core body 101, exposed areas 201 of the perimeter surface 117A are present. In one example, a back wall 113 is attached to a back portion 107 and a first side wall 111A is attached to a first side portion 105A. In the same example, the adjacent back wall 113 and first side wall 111A are each sized such that a predetermined exposed area 201 is included between a terminating edge of the back wall 113 and an adjacent terminating edge of the first side wall 111A. In various embodiments, the exposed areas 201 improve overall biocompatibility of the implant 100, by increasing a proportion of the implant 100 that is both in contact with bone and demonstrates a modulus of elasticity similar to bone.

In one or more embodiments, autologous bone and/or bone graft substitutes are included in one or more of the exposed areas 201 to enhance healing and osseointegration. In at least one embodiment, additives such as bioactive ceramics (e.g., silicates, hydroxyapatite, tricalcium phosphate, etc.), are included in one or more of the exposed areas 201 to enhance healing and osseointegration.

According to one embodiment, the core body 101 includes one or more macro-structural features. In at least one embodiment, the one or more macro-structural features include holes 203A, 203B configured for receiving insertion instruments, fixtures, etc. In various embodiments, the one or more macro-structural features of portions of the core body 101 are reflected in the construction of corresponding walls 109, 111A, 111B, and/or 113 attached to the portions. In one or more embodiments, the walls 109, 111A, 111B, and/or 113 include holes 205A, 205B that correspond to and are aligned with the holes 203A, 203B, respectively. According to one embodiment, reciprocity of macro-structural features between the core body 101 and walls 109, 111A, 111B, and/or 113 preserves implant functionality and workflows, such as, for example, implantation procedures. In various embodiments, the holes 203A, 203B advantageously permit radiographic evaluation of the implant 100 from lateral viewing angles.

FIG. 3 shows a top (e.g., superior) view of the implant 100. In at least one embodiment, the implant 100 includes a plurality of corners 301A-D. According to one embodiment, the plurality of corners 301A-D are rounded to reduce a risk of tissue damage or interference during and after implantation of the implant 100 to a target site. In one or more embodiments, the core body 101 includes the top surface 121 that is generally coplanar with a top surface 303 of each wall 109, 111A, 111B, and/or 113 attached to the core body 101.

In various embodiments, each wall 109, 111A, 111B, and/or 113 includes a thickness 303 between about 0.001-0.0394 inches, about 0.001-0.005 inches, about 0.005-0.010 inches, about 0.08 inches, about 0.010-0.015 inches, about 0.015-0.020 inches, about 0.020-0.025 inches, about 0.025-0.030 inches, about 0.030-0.035 inches, or about 0.035-0.040 inches. According to one embodiment, the thickness 303 is selected such that the implant 100 is sufficiently radiolucent (e.g., for radiographic imaging purposes). In at least one embodiment, a smaller thickness 303 is selected for implants for cervical spine-related applications and a greater thickness 303 is selected for implants for lumbar spine-related applications.

In at least one embodiment, the core body 101 tapers in width between the front portion 103 and the back portion 107. In at least one embodiment, the first and second side portions 105A, 105B are angled towards a center 302 of the core body 101. In various embodiments the angled side portions 105A, 105B transition the core body 101 from a first width 305 to a second width 307 measuring less than the first width 305. According to one embodiment, the first width 305 measures between about 5.0-40.0 mm, about 5.0 mm, about 5.0-10.0 mm, about 10.0-15.0 mm, about 15.0-20.0 mm, about 20.0-25.0 mm, about 25.0-30.0 mm, about 30.0-35.0 mm, about 35.0 mm, or about 35.0-40.0 mm. In at least one embodiment, the second width 307 measures between about 5.0-40.0 mm, about 5.0 mm, about 5.0-10.0 mm, about 10.0-15.0 mm, about 15.0-20.0 mm, about 20.0-25.0 mm, about 25.0-30.0 mm, about 30.0-35.0 mm, about 35.0 mm, or about 35.0-40.0 mm. In one or more embodiments, the first and second side portions 105A, 105B include one or more shapes including, but not limited to, cubes, rectangular prisms, and ovoids. In one embodiment, the first and second side portions 105A, 105B are curved and/or rounded towards the center 302 of the core body 101 (e.g., to provide a low profile thereto).

FIG. 4 shows a bottom (e.g., inferior) view of the implant 100. In at least one embodiment, as described herein, the core body 101 of the implant 100 includes a bottom surface 401. In one or more embodiments, the bottom surface 401 is generally coplanar with a bottom surface 403 of each wall 109, 111A, 111B, and/or 113 attached to the core body 101.

In at least one embodiment, the interior cavity 108 of the core body 101 includes a plurality of corners 405A-D. According to one embodiment, the plurality of corners 405A-D are rounded to reduce a risk of tissue damage or interference during and after implantation of the implant 100 to a target site. In various embodiments, the interior cavity 108 is filled with a bone growth-promoting material prior to implantation of the implant 100.

In various embodiments, the core body 101 includes a depth 407 (e.g., between the front portion 103 and the back portion 113). In one or more embodiments, the depth 407 measures between about 1.0-30.0 mm, about 1.0-3.0 mm, about 3.0-6.0 mm, about 5.0 mm, about 6.0-9.0 mm, about 9.0-12.0 mm, about 12.0-15.0 mm, about 15.0-18.0 mm, about 18.0-21.0 mm, about 21.0-24.0 mm, about 24.0 mm, about 24.0-27.0 mm, or about 27.0-30.0 mm.

FIG. 5 shows a front (e.g., anterior) view of the implant 100. In various embodiments, as discussed herein, the top surfaces 303 of the walls 109, 111A, 111B, and/or 113 are generally coplanar with the top surface 121 of the core body 101, and the bottom surfaces 403 of the walls 109, 111A, 111B, and/or 113 are generally coplanar with the bottom surface 121 of the core body 101. According to one embodiment, one or more of the top surfaces 303 are not coplanar with the top surface 121. In one example, a front wall 109 is sized and/or attached such that a top surface 303 thereof is inferior to a top surface 121 of a front portion 103 to which the front wall 109 is attached. In a similar example, a front wall 109 is sized and/or attached such that a bottom surface 403 thereof is superior to a bottom surface 401 of the front portion 103 to which the front wall 109 is attached. As will be understood by an individual of ordinary skill in the art, the heights of the top surfaces 303 and bottom surfaces 403 may deviate along lengths thereof to conform to height deviations of the corresponding top surfaces 121 and bottom surfaces 401.

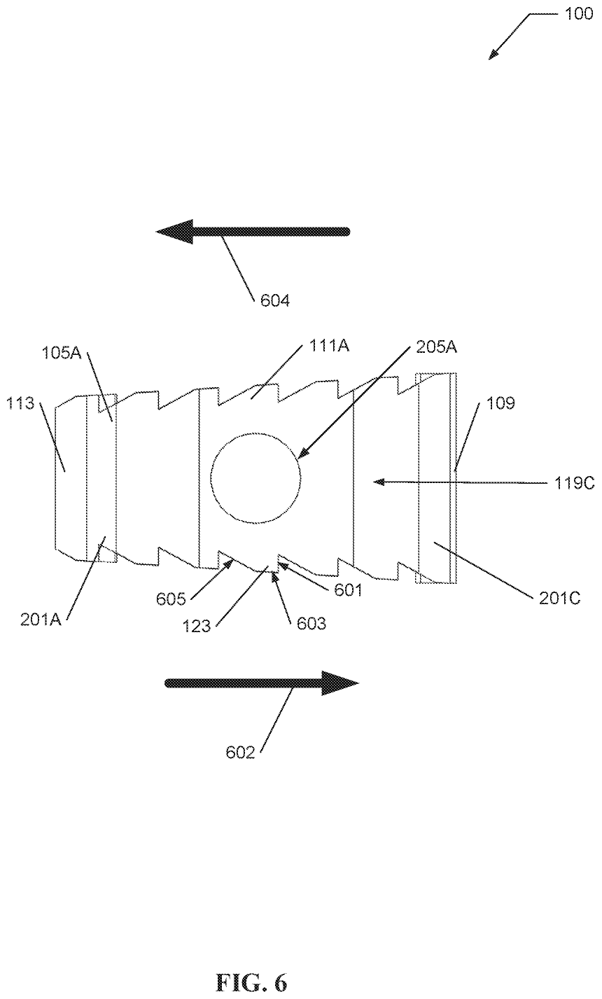

FIG. 6 shows a side (e.g., lateral) view of the implant 100. According to one embodiment, the implant 100 includes a plurality of expulsion resistance features 123 including ridges configured to resist pull-out forces (e.g., in a pull-out direction 602) that are experienced by the implant 100 at a target site. In various embodiments, the implant 100 includes between about 3-20 expulsion resistance features 123. As discussed herein, in at least one embodiment, the walls 109, 111A, 111B, and/or 113 include the expulsion resistance features 123. In one or more embodiments, an expulsion resistance feature 123 includes a first surface 601 oriented substantially perpendicular to a connected second surface 603 (e.g., that may form part of the top surface 121). In various embodiments, the first surface 601 is oriented substantially perpendicular to the pull-out direction 602 and, upon implantation, interfaces with tissue at the target site to restrict movement of the implant 100 in the pull-out direction 602, thereby resisting the pull-out forces discussed herein. According to one embodiment, the second surface 603 is connected, on an opposite end, to a third surface 605. In at least one embodiment, the third surface 605 is generally sloped towards an insertion direction 604 of the implant 100. In one or more embodiments, the general slope in toward the insertion direction 604 buttresses the expulsion resistance feature 123 against forces in the pull-out direction 602 and facilitates movement of the implant 100 in the insertion direction 604 because the sloped third surface 605 is less likely to catch on tissue during insertion at the target site.

FIG. 7 shows a side (e.g., lateral) view of the implant 100. In at least one embodiment, the core body 101 tapers in height between the front portion 103 and the back portion 107. In at least one embodiment, the side portions 105A (FIG. 1), 105B are angled towards a center 302 of the core body 101. In various embodiments, the angled side portions 105A, 105B transition the core body 101 from a first height 701 to a second height 704 measuring less than the first height 701. Accordingly, in various embodiments, the front wall 109, first and second side walls 111A, 111B, and back wall 113 include tapered shapes that conform to tapered shapes of the corresponding front portion 103, first and second side portions 105A, 105B, and back portion 107. According to one embodiment, the first height 701 measures between about 5.0-40.0 mm, about 5.0 mm, about 5.0-10.0 mm, about 10.0-15.0 mm, about 15.0-20.0 mm, about 20.0-25.0 mm, about 25.0-30.0 mm, about 30.0-35.0 mm, about 35.0 mm, or about 35.0-40.0 mm. In at least one embodiment, the second height 704 measures between about 5.0-40.0 mm, about 5.0 mm, about 5.0-10.0 mm, about 10.0-15.0 mm, about 15.0-20.0 mm, about 20.0-25.0 mm, about 25.0-30.0 mm, about 30.0-35.0 mm, about 35.0 mm, or about 35.0-40.0 mm.

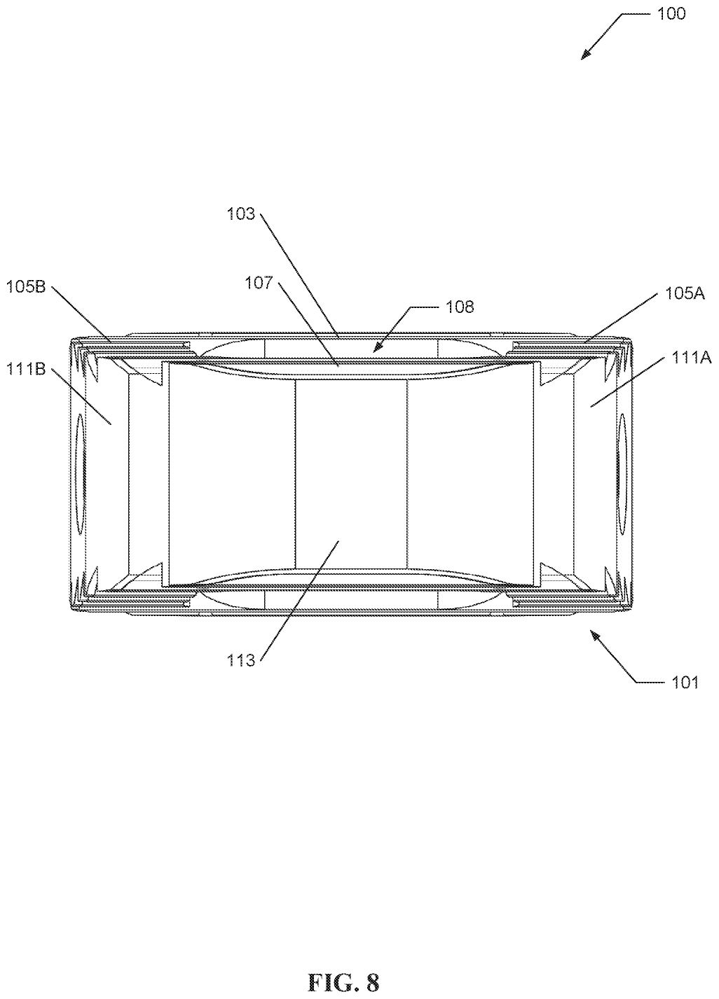

FIG. 8 shows a back (e.g., posterior) view of the implant 100.

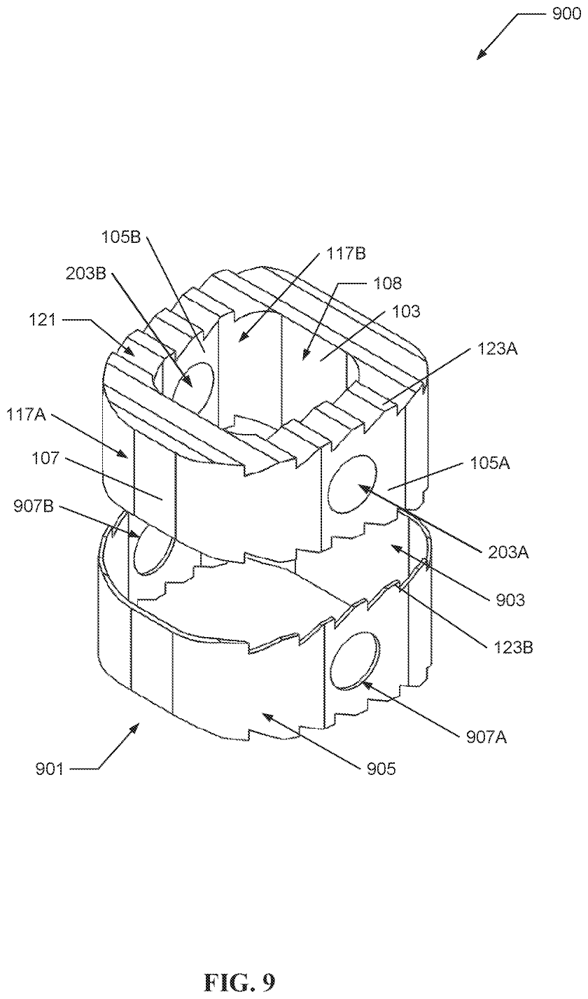

FIG. 9 shows an exploded view of an implant 900. In various embodiments, the implant 900 includes the core body 101 described herein. In at least one embodiment, the implant 900 includes an outer sleeve 901 that is attached over and to the core body 101. According to one embodiment, the attached outer sleeve 901 includes an interior surface 903 that is in conformed and substantially continuous contact with the perimeter surface 117A of the core body 101. In one or more embodiments the interior surface 903 substantially sheathes the perimeter surface 117A. In various embodiments, the shape of each portion of the interior surface 903 is specific to its intended attachment location on the perimeter surface 117A. Thus, in at least one embodiment, the specific geometry and dimensions of a target surface on the core body 101 forms a template for the specific geometry and dimensions of a corresponding portion of the interior surface 903 which contacts the target surface.

In one or more embodiments, the outer sleeve 901 includes an exterior surface 905. In various embodiments, the interior surface 903, the exterior surface 905, and/or portions of the outer sleeve 901 therebetween include one or more property modifications described herein for increasing ossification into the outer sleeve 901 and/or core body 101. According to one embodiment, the exterior surface 905 includes one or more surface features described herein for increasing ossification into the outer sleeve 901 and/or core body 101. In one example, the exterior surface 905 includes a plurality of porous structures permitting increased and more rapid osseointegration. In one or more embodiments, the exterior surface 905 demonstrates a porosity of at least about 60%, or between about 60-65%, about 65-70%, about 70-75%, about 75-80%, about 80-85%, about 85-90%, about 90-95%, or about 95-99%. In various embodiments, pores formed into the exterior surface 905, interior surface 903 (and/or other surfaces of the outer sleeve 901) include a width measuring between about 1-700 μm, about 250-300 μm, about 1-50 μm, about 50-100 μm, about 100-150 μm, about 150-200 μm, about 200-250 μm, about 250-300 μm, about 300, about 300-350 μm, about 350-400 μm, about 400-450 μm, about 450-500 μm, about 500-550 μm, about 550-600 μm, about 600-650 μm, about 650-700 μm, about 700 μm, about 700-750 μm. In at least one embodiment, a pore width between about 1-100 μm is selected, for example, to promote adhesion of osteoblasts to the implant 900.

In at least one embodiment, the interior surface 903, the exterior surface 905, and/or internal portions of the outer sleeve 901 are modified to demonstrate an increased porosity and/or an increased roughness (e.g., as compared to an unmodified embodiment thereof). Methods for increasing roughness and porosity, and for forming structures or pores on the interior surface 903, the exterior surface 905, and/or internal portions of the outer sleeve 901 may include, but are not limited to: 1) etching; 2) particle blasting; 3) micromachining by electrical techniques, electrochemical techniques, and otherwise; 4) coating; 5) selective laser melting (SLM); and 6) selective laser sintering (SLS).

In various embodiments, the one or more macro-structural features of portions of the core body 101 are reflected in the construction of corresponding portions of the outer sleeve 901 attached to the portions. In one or more embodiments, the outer sleeve 901 includes holes 907A, 907B that correspond to and are aligned with the holes 203A, 203B, respectively. According to one embodiment, reciprocity of macro-structural features between the core body 101 and outer sleeve 901 preserves implant functionality and workflows, such as, for example, implantation procedures. In various embodiments, the holes 907A, 907B advantageously permit radiographic evaluation of the implant 900 from lateral viewing angles.

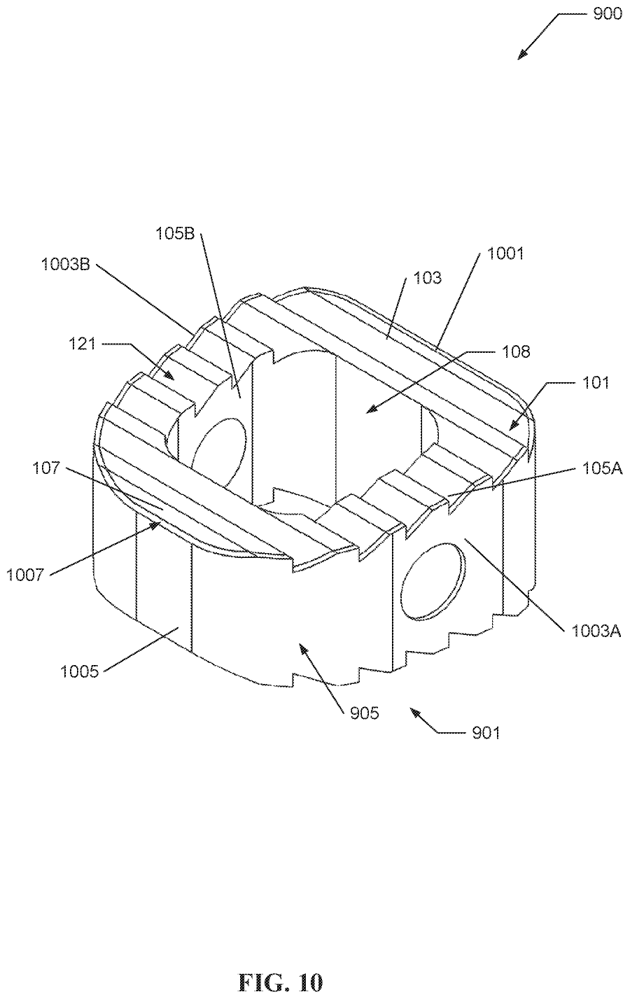

FIG. 10 shows a perspective view of the implant 900. In various embodiments, the outer sleeve 901 includes one or more enhancement materials described herein. In at least one embodiment, the outer sleeve 901 includes: 1) a front portion 1001 configured (e.g., sized and shaped) for attachment to the front portion 103; 2) a first side portion 1003A for attachment to the first side portion 105A); 3) a second side portion 1003B configured for attachment to the second side portion 105B; and 4) a back portion 1005 configured for attachment to the back portion 107. According to one embodiment, the outer sleeve 901 includes the interior surface 903 and an exterior surface 905. In one or more embodiments, the interior surface 903 of each portion of the outer sleeve 901 is formed to conform to a shape of the corresponding portion of the perimeter surface 117A (FIG. 1) with which the interior surface 903 interfaces. In one example, a perimeter surface 117A of a first side portion 105A includes a particular curved shape. In the same example, an interior surface 903 of a first side portion 1003A attached to the first side portion 105A includes a curved shape substantially similar to the particular curved shape of the perimeter surface 117A, thereby permitting conformed and substantially continuous contact therebetween.

According to one embodiment, the implant 900 is configured such that: 1) the front portion 1001 is attached to, is in conformed and substantially continuous contact with, and generally sheathes the perimeter surface 117A (not shown) of the front portion 103; 2) the first side portion 1003A is attached to, is in conformed and substantially continuous contact with, and generally sheathes the perimeter surface 117A of the first side portion 105A; 3) the second side portion 1003B is attached to, is in conformed and substantially continuous contact with, and generally sheathes the perimeter surface 117A of the second side portion 105B; and 4) the back portion 1005 is attached to, is in conformed and substantially continuous contact with, and generally sheathes the perimeter surface 117A of the back portion 107.

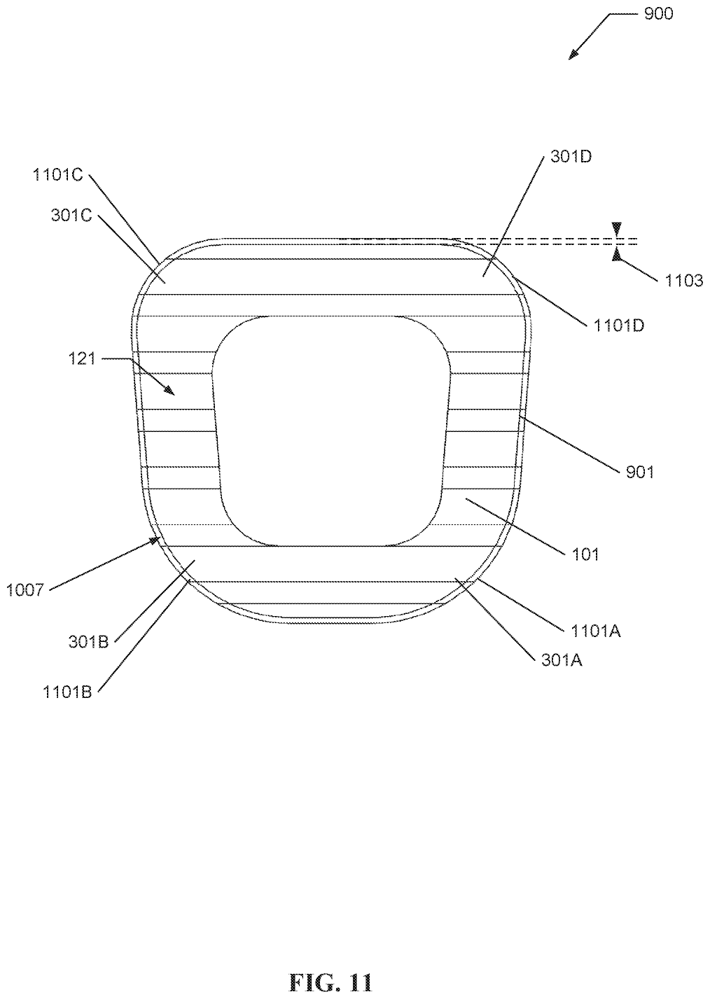

FIG. 11 shows a top (e.g., superior) view of the implant 900. According to one embodiment, the outer sleeve 901 includes one or more corners 1101. In at least one embodiment, the one or more corners 1101 are shaped substantially similar to corresponding corners 301 of the core body 101 to which the outer sleeve 901 is attached. In various embodiments, the outer sleeve 901 includes: 1) a corner 1101A that is shaped substantially similar to the corner 301A; 2) a corner 1101B that is shaped substantially similar to the corner 301B; 3) a corner 1101C that is shaped substantially similar to the corner 301C; and 4) a corner 1101D that is shaped substantially similar to the corner 301D. In one or more embodiments, the corners 1101 are rounded or curved to reduce a risk of the implant 900 becoming caught on, piercing, or lacerating tissue at a target site.

In one or more embodiments, the outer sleeve 901 includes a top surface 1007 that is generally coplanar with a top surface 121 of the core body 101. In various embodiments, the outer sleeve 901 includes a thickness 1103 between about 0.001-0.0394 inches, about 0.001-0.005 inches, about 0.005-0.010 inches, about 0.08 inches, about 0.010-0.015 inches, about 0.015-0.020 inches, about 0.020-0.025 inches, about 0.025-0.030 inches, about 0.030-0.035 inches, or about 0.035-0.040 inches. According to one embodiment, the thickness 1103 is selected such that the implant 900 is sufficiently radiolucent (e.g., for radiographic imaging purposes). In at least one embodiment, a smaller thickness 1103 is selected for implants for cervical spine-related applications and a greater thickness 1103 is selected for implants for lumbar spine-related applications.

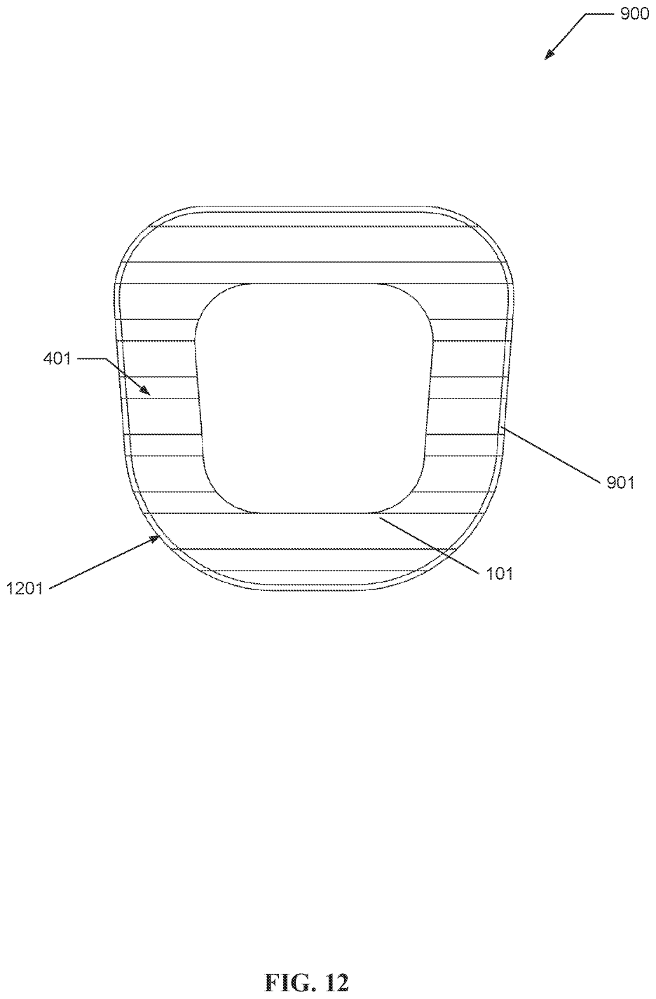

FIG. 12 shows a bottom (e.g., inferior) view of the implant 900. In one or more embodiments, the bottom surface 401 of the core body 101 is generally coplanar with a bottom surface 1201 of the outer sleeve 901 attached to the core body 101.

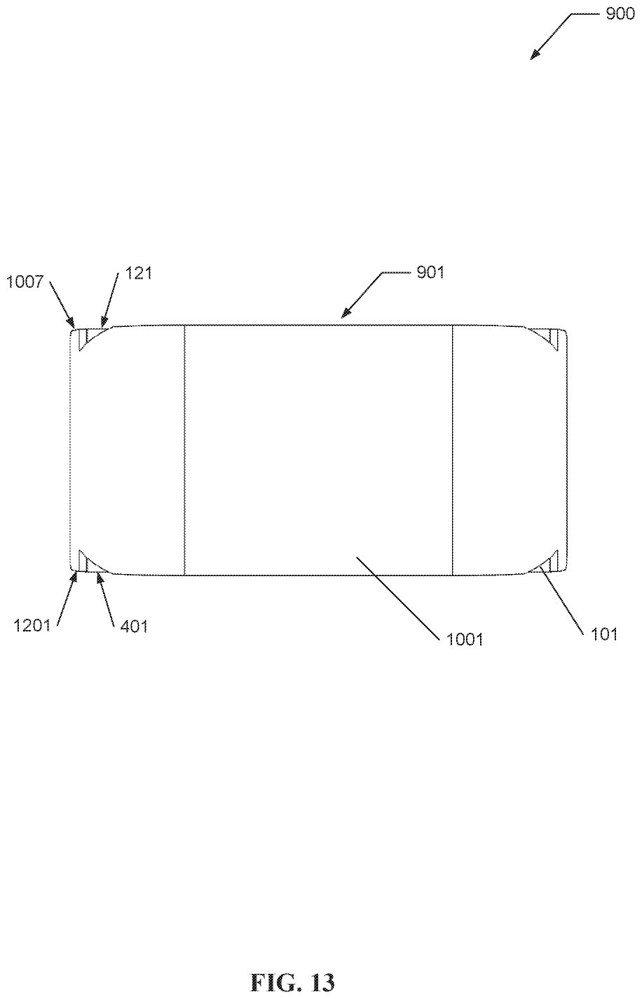

FIG. 13 shows a front (e.g., anterior) view of the implant 900. In various embodiments, as discussed herein, the top surface 1007 of the outer sleeve 901 is generally coplanar with of the top surface 121 of corresponding portions of the core body 101, and the bottom surface 1201 of the outer sleeve 901 is generally coplanar with the bottom surface 401 of corresponding portions of the core body 101. In various embodiments, portions of the outer sleeve 901 are shaped such that the top surface 1007 and bottom surface 1201 thereof conform to angles of corresponding front portions 103, side portions 105A, 105B, and back portions 107 of the core body 101.

In one example, the implant 900 is a spinal implant and includes a core body 101 with front portions 103, side portions 105A, 105B, and back portions 107 shaped according to a predetermined lordotic angle. In the same example, portions of a corresponding an attached outer sleeve 901 are shaped to conform to the front portions 103, side portions 105A, 105B, and back portions 107 and according to the predetermined lordotic angle. In a second example, a wedge implant includes a core body shaped according to predetermined osteotomy angles. In the same example, a corresponding outer sleeve of enhancement material attached to the core body is shaped to conform thereto and according to the predetermined osteotomy angles.

According to one embodiment, one or more portions of the top surface 1007 are not coplanar with the top surface 121. In one example, a front portion 1001 is sized and/or attached such that a top surface 1007 thereof is inferior to a top surface 121 of a front portion 103 (not shown) to which the front portion 1001 is attached. In a similar example, a front portion 1001 is sized and/or attached such that a bottom surface 403 thereof is superior to a bottom surface 1201 of the front portion 103 to which the front portion 1001 is attached. As will be understood by an individual of ordinary skill in the art, the heights of the top surface 1007 and bottom surface 1203 may deviate along lengths thereof to conform to height deviations of the corresponding top surfaces 121 and bottom surfaces 401 of the core body 101.

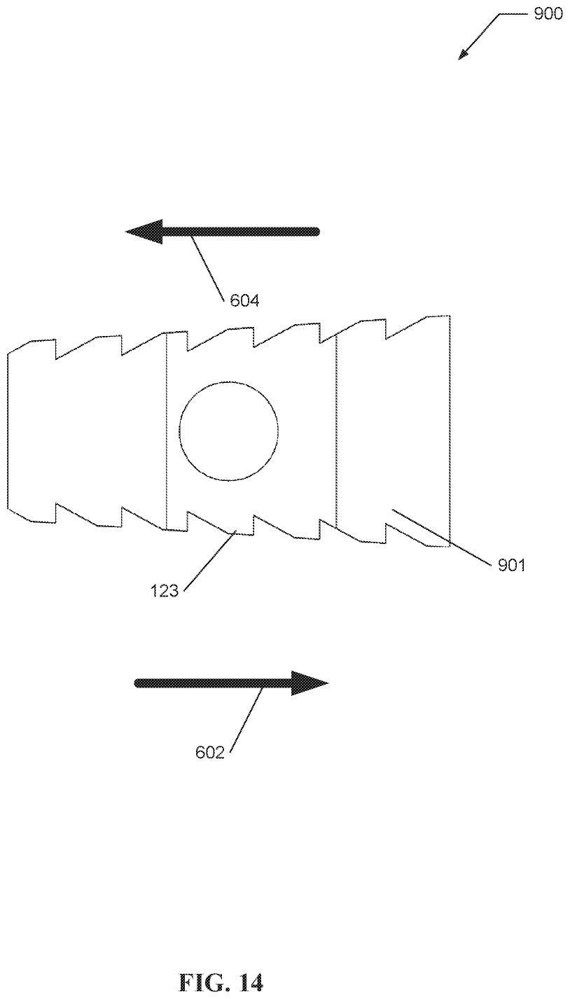

FIG. 14 shows a side (e.g., lateral) view of the implant 900. According to one embodiment, the outer sleeve 901 includes one or more of the expulsion resistance features 123B, thereby providing the implant 900 resistance to pull-out forces (e.g., in the direction 602) and facilitating insertion of the implant 900 (e.g., in the direction 604) to a target site.

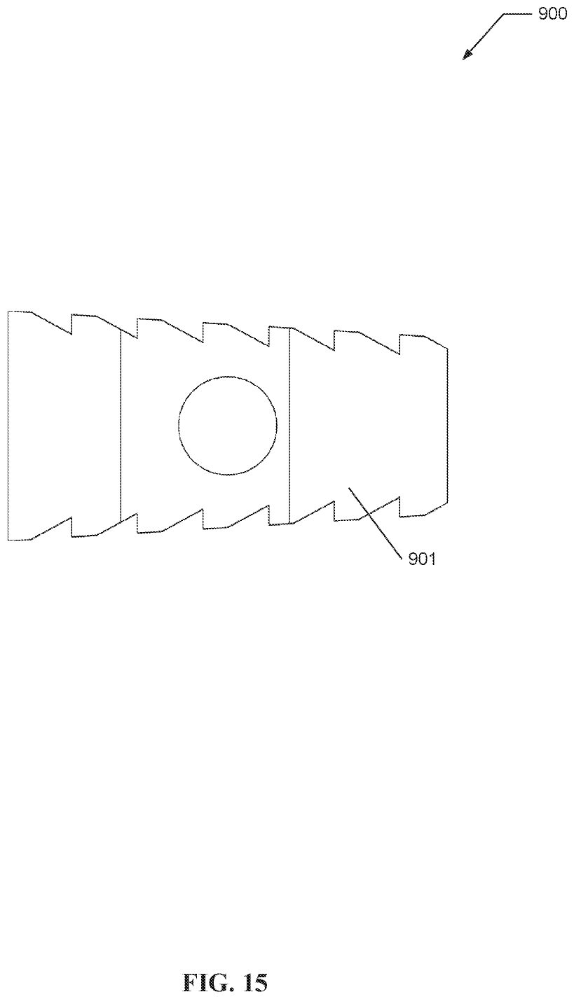

FIG. 15 shows a side (e.g., lateral) view of the implant 900.

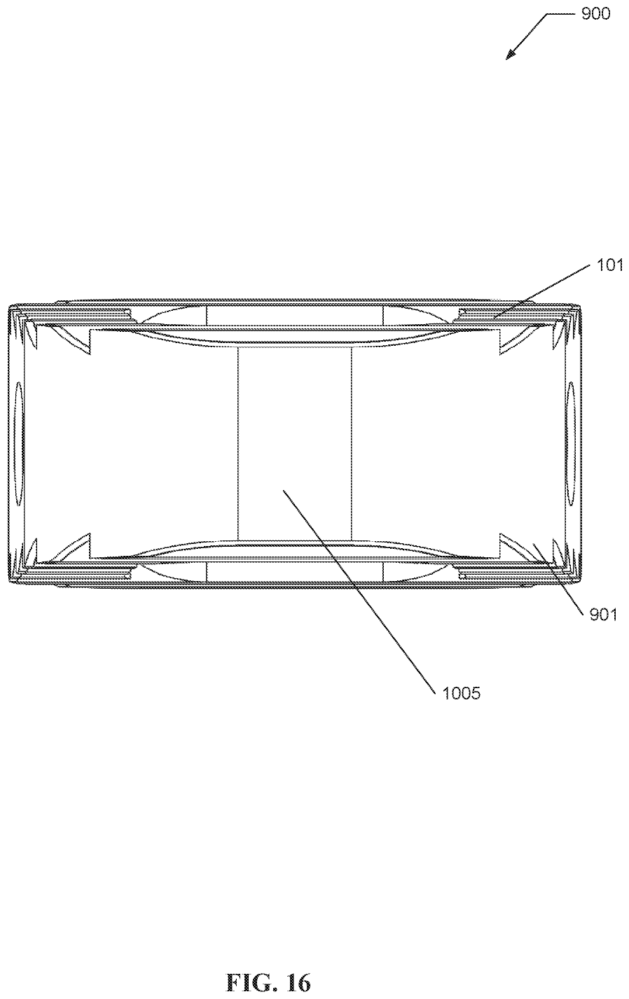

FIG. 16 shows a back (e.g., posterior) view of the implant 900.

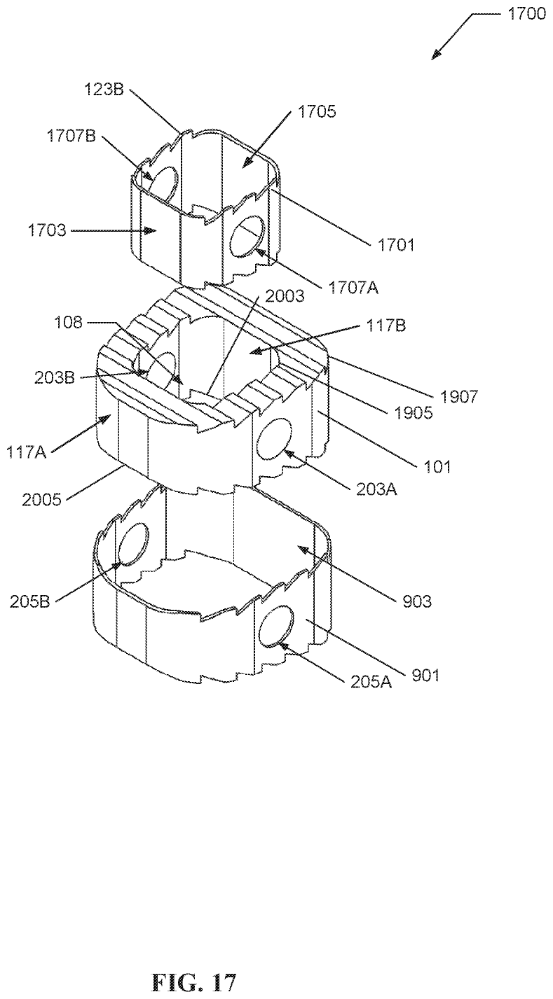

FIG. 17 shows an exploded view of an implant 1700. In various embodiments, the implant 1700 includes the core body 101 and outer sleeve 901 configured as described herein (e.g., substantially identical to the implant 900). In at least one embodiment, the implant 1700 includes an inner sleeve 1701 that is attached to the core body 101 within the interior cavity 108. According to one embodiment, the attached inner sleeve 1701 includes an exterior surface 1703 that is in conformed and substantially continuous contact with the perimeter surface 117B of the core body 101. In one or more embodiments the exterior surface 1703 substantially sheathes the perimeter surface 117B. In various embodiments, the shape of each portion of the exterior surface 1703 is specific to its intended attachment location on the perimeter surface 117B. Thus, in at least one embodiment the specific geometry and dimensions of a target surface on the core body 101 forms a template for the specific geometry and dimensions of a corresponding portion of the exterior surface 1703 which contacts the target surface.

In one or more embodiments, the inner sleeve 1701 includes an interior surface 1705. In various embodiments, the interior surface 1705, exterior surface 1703, and/or internal portions of the inner sleeve 1701 include one or more property modifications described herein for increasing ossification into the inner sleeve 1701 and/or core body 101. In one example, the interior surface 1705 includes a plurality of porous structures permitting increased and more rapid osseointegration. In one or more embodiments, the interior surface 1705 demonstrates a porosity of at least about 60%, or between about 60-65%, about 65-70%, about 70-75%, about 75-80%, about 80-85%, about 85-90%, about 90-95%, or about 95-99%. In various embodiments, pores formed into the exterior surface 1703, interior surface 1705 (and/or other surfaces of the inner sleeve 1701) include a width measuring between about 1-700 μm, about 250-300 μm, about 1-50 μm, about 50-100 μm, about 100-150 μm, about 150-200 μm, about 200-250 μm, about 250-300 μm, about 300, about 300-350 μm, about 350-400 μm, about 400-450 μm, about 450-500 μm, about 500-550 μm, about 550-600 μm, about 600-650 μm, about 650-700 μm, about 700 μm, about 700-750 μm. In at least one embodiment, a pore width between about 1-100 μm is selected, for example, to promote adhesion of osteoblasts to the implant 1700.

In at least one embodiment, the interior surface 1705, exterior surface 1703, and/or internal portions of the inner sleeve 1701 are modified to demonstrate an increased porosity and an increased roughness (e.g., as compared to an unmodified embodiment thereof). Methods for increasing roughness and porosity, and for forming structures or pores on the interior surface 1705, exterior surface 1703, and/or internal portions of the inner sleeve 1701 may include, but are not limited to: 1) etching; 2) particle blasting; 3) micromachining by electrical techniques, electrochemical techniques, and otherwise; 4) coating; 5) selective laser melting (SLM); and 6) selective laser sintering (SLS).

According to one embodiment, the inner sleeve 1701 includes one or more of the expulsion resistance features 123B, thereby providing the implant 1700 resistance to pull-out forces (e.g., in the direction 602, FIG. 6) and facilitating insertion of the implant 1700 (e.g., in the direction 604, FIG. 6) to a target site.

In various embodiments, the one or more macro-structural features of portions of the core body 101 are reflected in the construction of corresponding portions of the inner sleeve 1701 attached to the portions. In one or more embodiments, the inner sleeve 1701 includes holes 1707A, 1707B that correspond to and are aligned with the holes 203A, 203B, respectively. According to one embodiment, reciprocity of macro-structural features between the core body 101, outer sleeve 901, and inner sleeve 1701 preserves implant functionality and workflows, such as, for example, implantation procedures. In various embodiments, the holes 1707A, 1707B advantageously permit radiographic evaluation of the implant 1700 from lateral viewing angles.

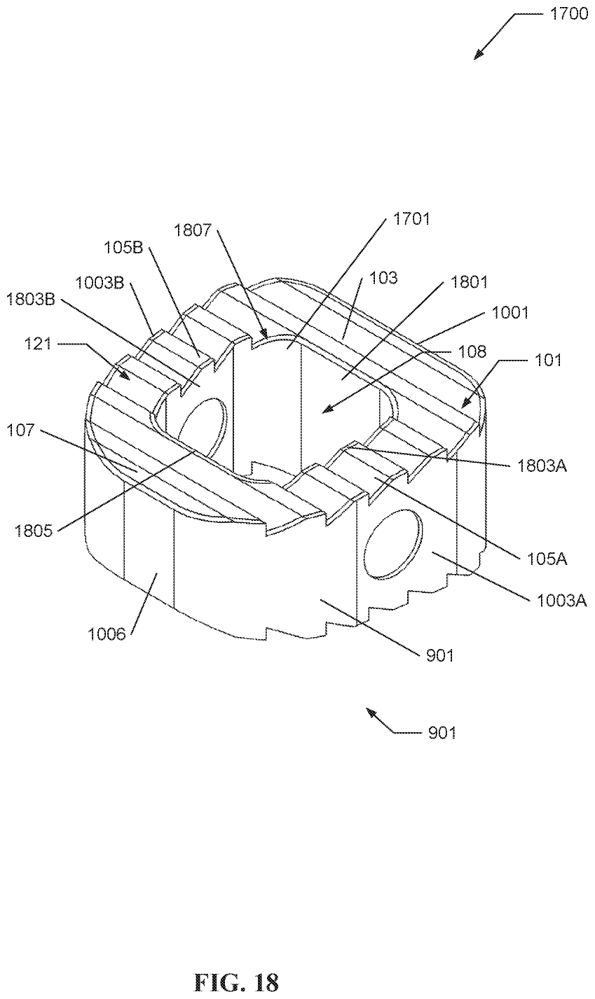

FIG. 18 shows a perspective view of the implant 1700. In various embodiments, the inner sleeve 1701 includes one or more enhancement materials described herein. In at least one embodiment, the inner sleeve 1701 includes: 1) a front portion 1801 configured (e.g., sized and shaped) for attachment to the front portion 103 (e.g., on the side thereof that includes the perimeter surface 117B); 2) a first side portion 1803A for attachment to the first side portion 105A (e.g., on the perimeter surface 117B side); 3) a second side portion 1803B configured for attachment to the second side portion 105B (e.g., on the perimeter surface 117B side); and 4) a back portion 1805 configured for attachment to the back portion 107 (e.g., on the perimeter surface 117B side). In one or more embodiments, the exterior surface 1703 of each portion of the inner sleeve 1701 is formed to conform to a shape of the corresponding portion of the perimeter surface 117B (FIG. 1) with which the exterior surface 1703 interfaces. In one example, a perimeter surface 117B of a back portion 107 includes a particular curved shape. In the same example, an exterior surface 1703 of a back portion 1805 attached to the back portion 107 includes a curved shape substantially similar to the particular curved shape of the perimeter surface 117B, thereby permitting conformed and substantially continuous contact therebetween.

According to one embodiment, the implant 1700 is configured such that: 1) the front portion 1001 is attached to, is in conformed and substantially continuous contact with, and generally sheathes the perimeter surface 117A (not shown) of the front portion 103; 2) the first side portion 1003A is attached to, is in conformed and substantially continuous contact with, and generally sheathes the perimeter surface 117A of the first side portion 105A; 3) the second side portion 1003B is attached to, is in conformed and substantially continuous contact with, and generally sheathes the perimeter surface 117A of the second side portion 105B; 4) the back portion 1005 is attached to, is in conformed and substantially continuous contact with, and generally sheathes the perimeter surface 117A of the back portion 107; 5) the front portion 1801 is attached to, is in conformed and substantially continuous contact with, and generally sheathes the perimeter surface 117B (not shown) of the front portion 103; 6) the first side portion 1803A is attached to, is in conformed and substantially continuous contact with, and generally sheathes the perimeter surface 117B of the first side portion 105A; 7) the second side portion 1803B is attached to, is in conformed and substantially continuous contact with, and generally sheathes the perimeter surface 117B of the second side portion 105B; and 8) the back portion 1805 is attached to, is in conformed and substantially continuous contact with, and generally sheathes the perimeter surface 117B of the back portion 107.

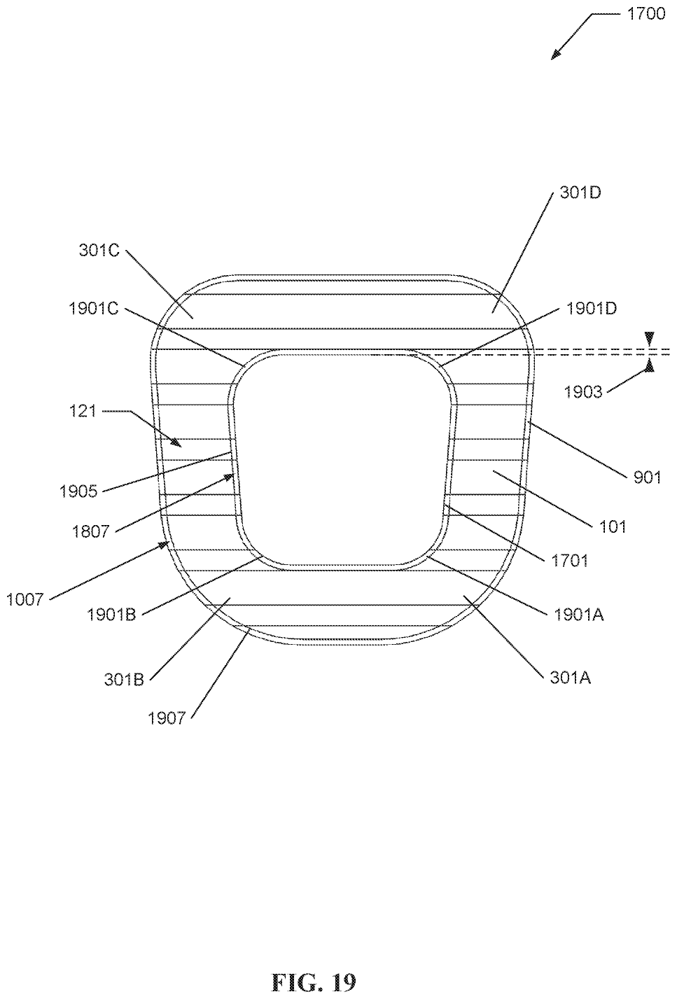

FIG. 19 shows a top (e.g., superior) view of the implant 1700. According to one embodiment, the inner sleeve 1701 includes one or more corners 1901. In at least one embodiment, the one or more corners 1901 are shaped substantially similar to corresponding corners 301 of the core body 101 to which the inner sleeve 1701 is attached. In various embodiments, the inner sleeve 1701 includes: 1) a corner 1901A that is shaped substantially similar to the corner 301A; 2) a corner 1901B that is shaped substantially similar to the corner 301B; 3) a corner 1901C that is shaped substantially similar to the corner 301C; and 4) a corner 1901D that is shaped substantially similar to the corner 301D. In one or more embodiments, the corners 1901 are rounded or curved to reduce a risk of the implant 1700 becoming caught on, piercing, or lacerating tissue at a target site.

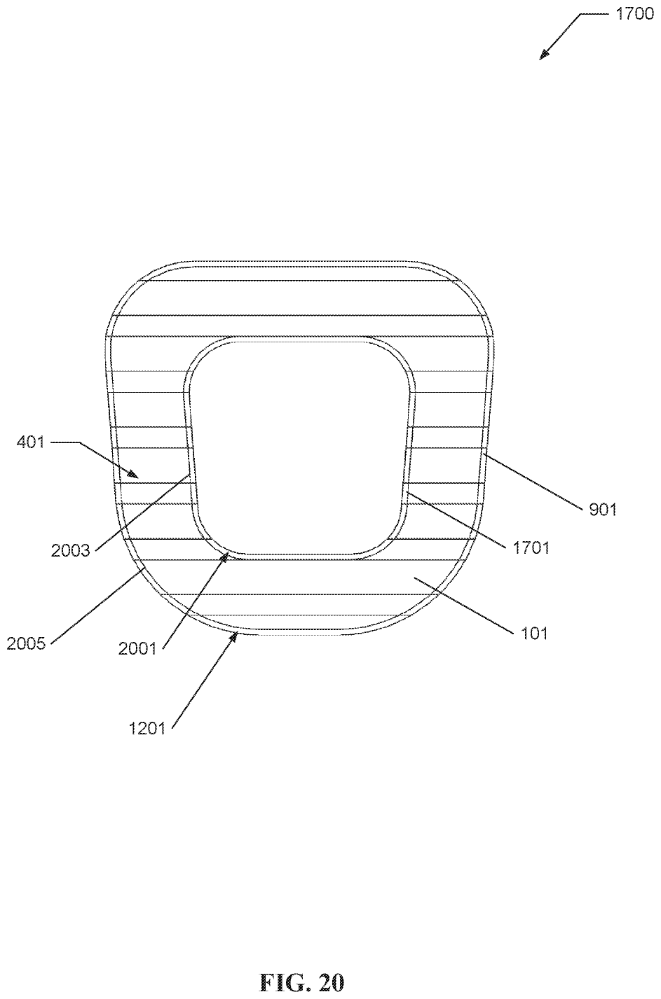

In one or more embodiments, the inner sleeve 1701 includes a top surface 1807 that is generally coplanar with the top surface 121 of the core body 101 and/or the top surface 1007 of the outer sleeve 901. In various embodiments, as discussed herein, the top surface 1807 of the inner sleeve 1701 is generally coplanar with of the top surface 121 of corresponding portions of the core body 101, and the bottom surface 2001 (FIG. 20) of the inner sleeve 1701 is generally coplanar with the bottom surface 401 of corresponding portions of the core body 101. In various embodiments, portions of the inner sleeve 1701 are shaped such that the top surface 1807 and bottom surface 2001 thereof conform to angles of corresponding front portions 103, side portions 105A, 105B, and back portions 107 of the core body 101.

In one example, the implant 1700 is a spinal implant and includes a core body 101 with front portions 103, side portions 105A, 105B, and back portions 107 shaped according to a predetermined lordotic angle. In the same example, portions of a corresponding an attached inner sleeve 1701 are shaped to conform to the front portions 103, side portions 105A, 105B, and back portions 107 and according to the predetermined lordotic angle. In a second example, a wedge implant includes a core body shaped according to predetermined osteotomy angles. In the same example, a corresponding inner sleeve of enhancement material attached to the core body is shaped to conform thereto and according to the predetermined osteotomy angles.

In various embodiments, the inner sleeve 1701 includes a thickness 1903 between about 0.001-0.0394 inches, about 0.001-0.005 inches, about 0.005-0.010 inches, about 0.08 inches, about 0.010-0.015 inches, about 0.015-0.020 inches, about 0.020-0.025 inches, about 0.025-0.030 inches, about 0.030-0.035 inches, or about 0.035-0.040 inches. According to one embodiment, the thickness 1903 is selected such that the implant 1700 is sufficiently radiolucent (e.g., for radiographic imaging purposes). In at least one embodiment, a smaller thickness 1903 is selected for implants for cervical spine-related applications and a greater thickness 1903 is selected for implants for lumbar spine-related applications.

In at least one embodiment, the top surface 121 includes a top surface inner edge 1905 and a bottom surface inner edge 2003 (FIG. 20). In one or more embodiments, the area between the top surface inner edge 1905 and the bottom surface inner edge 2003 includes the perimeter surface 117B (FIG. 1). In at least one embodiment, the top surface 121 includes a top surface outer edge 1907 and a bottom surface outer edge 2005 (FIG. 20). In one or more embodiments, the area between the top surface outer edge 1907 and the bottom surface outer edge 2005 includes the perimeter surface 117A (FIG. 1).

FIG. 20 shows a bottom (e.g., inferior) view of the implant 1700. In one or more embodiments, the bottom surface 401 of the core body 101 is generally coplanar with a bottom surface 2001 of the inner sleeve 1701 attached to the core body 101 and/or with the bottom surface 1201 of the outer sleeve 901.

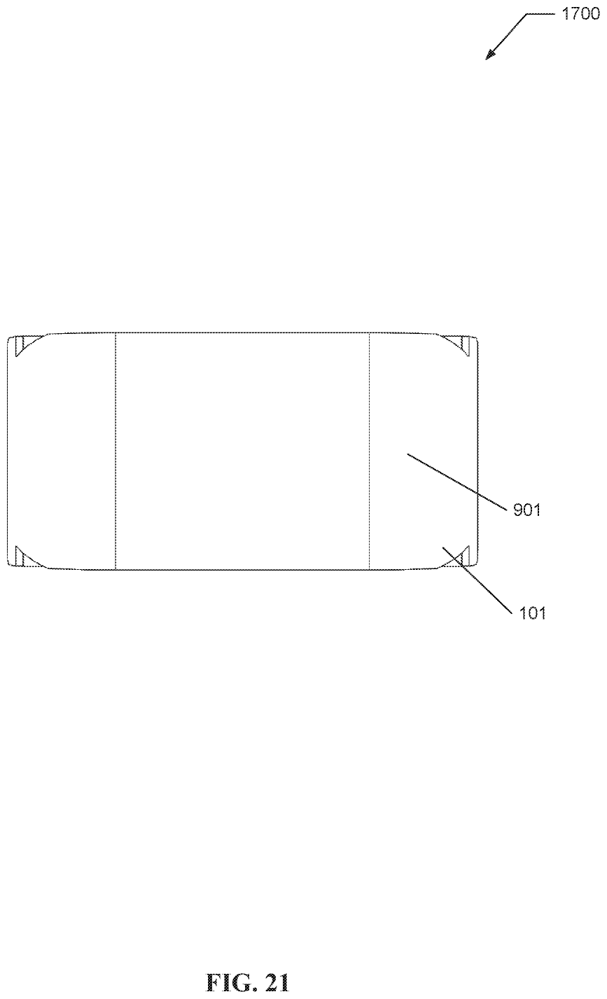

FIG. 21 shows a front (e.g., anterior) view of the implant 1700.

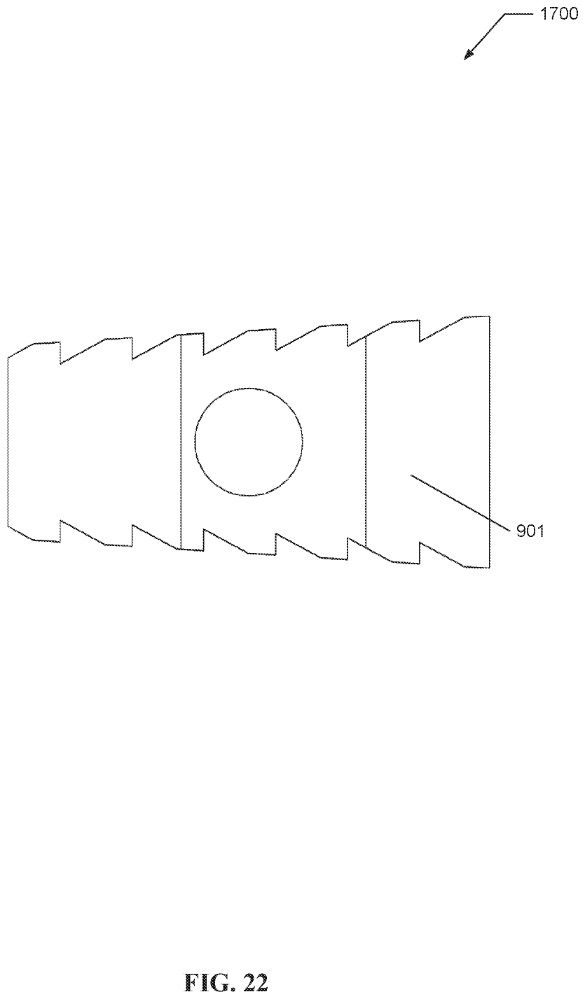

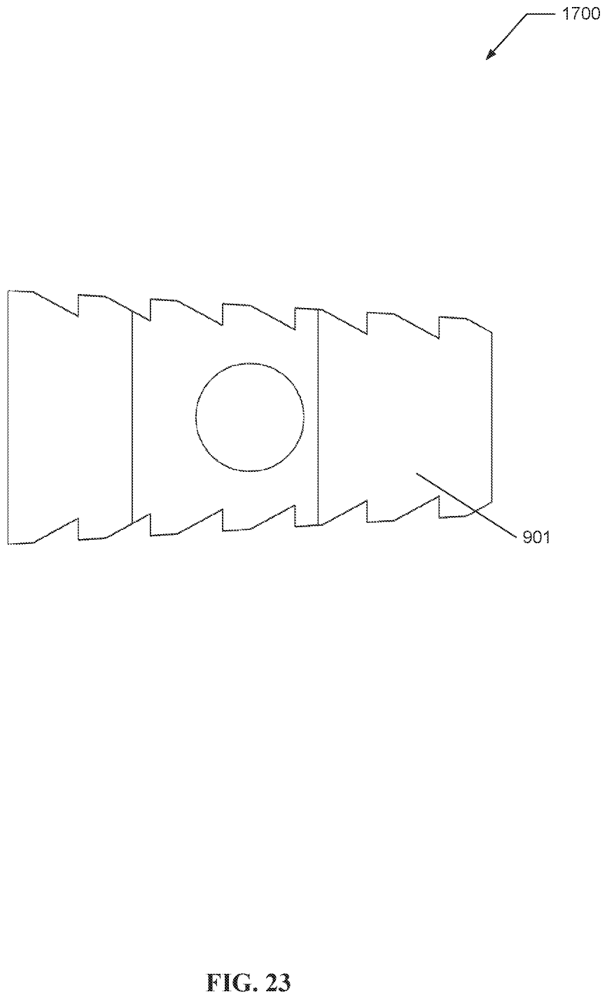

FIGS. 22-23 show side (e.g., lateral) views of the implant 1700.

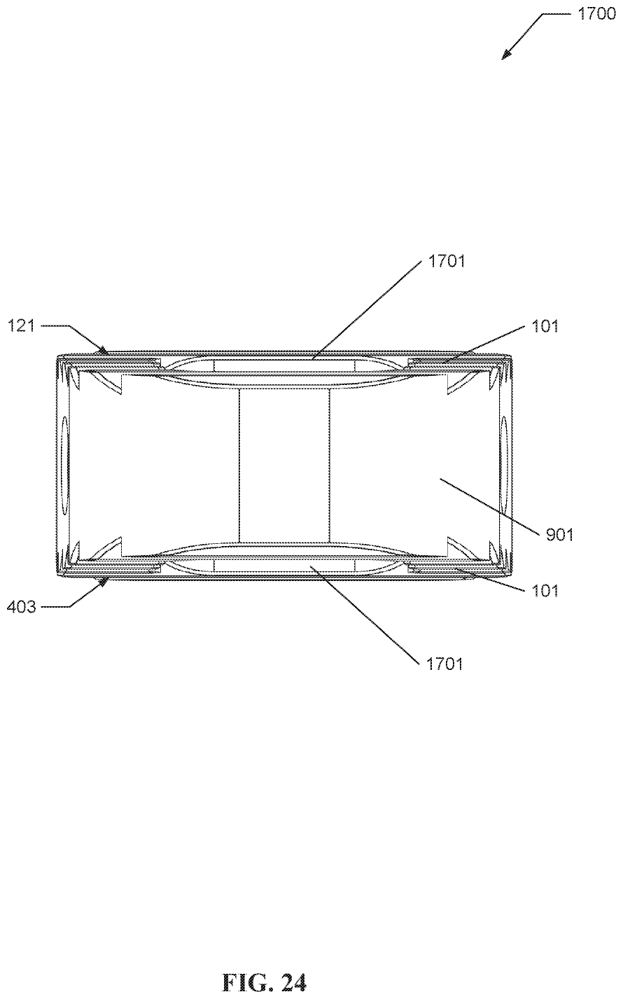

FIG. 24 shows a back (e.g., posterior) view of the implant 1700.

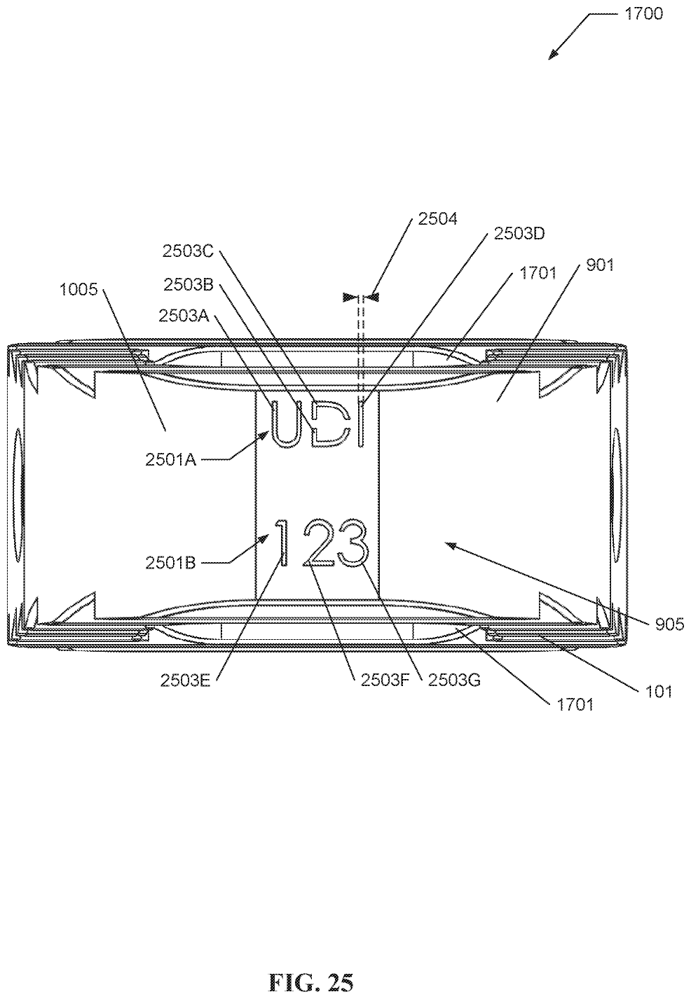

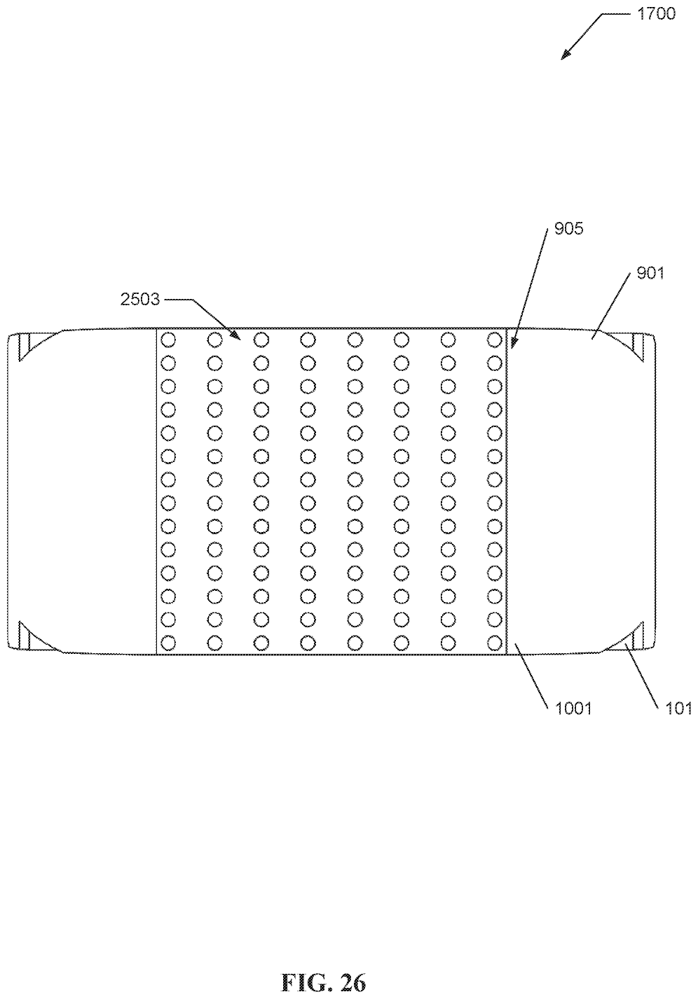

FIGS. 25-26 show an exemplary implant 1700 with encoded information. In various embodiments, any implant described herein includes the radiolucent properties of PEEK core material to encode meaningful information via subtraction of specific sections of a radiopaque enhancement material (e.g., titanium). As will be understood from discussions herein, PEEK and/or other core body 101 materials may not produce significant artifacts on outputs of imaging modalities including, but not limited to, magnetic resonance imaging (MRI), computed tomography (CT), multidetector-row computed tomography (MDCT), and X-ray. In contrast, an enhancement material of a wall 109, 111A, 111B, 113, outer sleeve 901, and/or inner sleeve 1701 may produce distinguishable artifacts in outputs of imaging modalities. In various embodiments, the subtraction of radiopaque material against a radiolucent material in specific areas and in specific patterns may enable the discernment of such structures in the output images of imaging modes and, by this process, meaningful information may be encoded. In various embodiments, the surface modifications to a wall 109, 111A, 111B, and/or 113, outer sleeve 901, and/or inner sleeve 1701 for information encoding may be achieved via a variety of material subtractive techniques including, but not limited to: 1) etching; 2) laser marking; 3) laser machining; and 4) micromachining by electrical techniques, electrochemical techniques and otherwise.

According to one embodiment, the encoded information includes, but is not limited to: 1) unique device identification (UDI); 2) manufacturing and production tracking numbers; 3) patient-specific information; 4) implant-specific information; and 5) procedure-specific information. In various embodiments, the encoded information is encoded in one or more formats including, but not limited to: 1) raw text; 2) one or more pore matrices; 3) QR codes; 4) bar codes; 5) geometric patterns; and 6) any other coded or uncoded text, numbers, or the like.

FIG. 25 shows a back (e.g., posterior) view of the implant 1700. In one or more embodiments, the back portion 1005 of the outer sleeve 901 includes encoded information 2501A, 2501B. In at least one embodiment, the encoded information 2501A, 2501B is a unique device identification (e.g., “UDI 123”). In at least one embodiment, the encoded information 2501A, 2501B is formed from a plurality of voids 2503 formed into the outer sleeve 901. According to one embodiment, the encoded information 2501A is encoded by the voids 2503A-D and the encoded information 2501B is encoded by the voids 2503E-G. In one or more embodiments, the voids 2503 penetrate from the external surface 905 through the internal surface 903 (not shown) of the outer sleeve 901. In other words, in at least one embodiment, the voids 2503 pass fully through the outer sleeve 901 and expose portions of the core body 101.

According to one embodiment, portions of the outer sleeve 901, inner sleeve 1701, and/or walls 109, 111A, 111B, and/or 113 are selectively thickened to increase a radiologic signature thereof and, thereby, improve discernment of the encoded information 2501 formed by the voids 2503. In at least one embodiment, only small areas (e.g., near the encoded information 2501) of a portion of the outer sleeve 901, inner sleeve 1701, and/or walls 109, 111A, 111B, and/or 113 are thickened. In various embodiments, thickening only the small areas, as opposed to thickening an entire portion of an implant, minimizes increases in the implant's overall radiologic signature. According to one embodiment, minimization of the implant's overall radiologic signature may reduce interference in radiative imaging, such as magnetic resonance imaging (MRI), and reduce obscuring of bone healing evidence in radiographs produced therefrom.

In various embodiments, upon the implant 1700 being imaged by one or more radiative imaging techniques, the outer sleeve 901 generates image artifacts (e.g., and is thus radiopaque), but the core body 101 does not generate substantial image artifacts (e.g., and is thus radiolucent). According to one embodiment, the subtraction of radiopaque material against a radiolucent material in the voids 2503 enables the discernment of the encoded information 2501A, 2501B in the output imagery generated by the one or more radiative imaging techniques.

According to one embodiment, the voids 2503 include a width 2504. In one or more embodiments, the width 2504 measures between about 300-700 μm, about 250-300 μm, about 300, about 300-350 μm, about 350-400 μm, about 400-450 μm, about 450-500 μm, about 500-550 μm, about 550-600 μm, about 600-650 μm, about 650-700 μm, about 700 μm, about 700-750 μm.

FIG. 26 shows a front (e.g., anterior) view of the implant 1700. According to one embodiment, the front portion 1001 of the outer sleeve 901 includes encoded information 2501. In at least one embodiment, the encoded information 2501 is encoded by a plurality of voids 2503 formed into the outer sleeve 901. In at least one embodiment, the plurality of voids 2503 are formed in a particular arrangement, such as a dot matrix or another matrix type, that codifies the encoded information 2501. In at least one embodiment, upon the implant 1700 being imaged by one or more radiative imaging techniques, the plurality of voids 2503 results in image artifact subtraction that renders the encoded information 2501 discernable in output of the one or more radiative imaging techniques.

### Additional Description of Exemplary Embodiments

It will be understood by an individual of ordinary skill in the art that various embodiments of the implants described herein included combinations of the present walls and sleeves. In one example, an implant includes a core body 101 in which an attached outer sleeve 901 generally sheathes the perimeter surface 117A and one or more attached walls 109, 111A, 111B, and/or 113 generally sheathe the perimeter surface 117B. In a second example, an implant includes a core body 101 in which one or more attached walls 109, 111A, 111B, and/or 113 generally sheathe the perimeter surface 117A and an attached inner sleeve 1701 generally sheathes the perimeter surface 117B. In a third example, an implant includes a core body 101 in which one or more attached walls 109, 111A, 111B, and/or 113 generally sheathe the perimeter surface 117B. In a fourth example, an implant includes a core body 101 in which an attached inner sleeve 1701 generally sheathes the perimeter surface 117B.

In one or more embodiments, the walls, outer sleeves, and/or inner sleeves of the present implants include surface and/or sub-surface modifications for promoting osseointegration. According to one embodiment, the walls, outer sleeves, and/or inner sleeves include pore and/or other roughness features on a top surface and/or a bottom surface. In a first example, a front wall 109 includes pore structures on a top surface 303. In a second example, an outer sleeve 901 includes pore structures on both a top surface 1007 and a bottom surface 1201. In a third example, an inner sleeve 1701 includes roughness features on a bottom surface 2001.

In one or more embodiments, the walls, outer sleeves, and inner sleeves described herein include varying thicknesses. In one example, a back wall 113 includes a greater thickness towards a top surface 303 and a lower thickness towards a bottom surface 403. In a second example, an outer sleeve 901 includes an increased thickness in first and second side portions 1003A, 1003B thereof and a reduced thickness in a front portion 1001 and a back portion 1005 thereof. In various embodiments, by varying the thickness of the of the enhancement material, the amount of load sharing with the core body is altered. According to one embodiment, because cells (e.g., osteoblasts and their precursors) respond favorably to a modest amount of stress loading and negatively either too much stress shielding or too much load, the thickness and macro-structure of the enhancement material and/or surface or sub-surface structures thereof (such as pores and structures providing roughness) are changed to optimize stress sharing with the core body (e.g., the primary load bearing structure).

In various embodiments, the thicknesses of walls, inner sleeves, and/or outer sleeves are varied to create an anisotropic or isotropic (e.g., in the case of equal thicknesses) property in the implant. In one example, walls of an implant demonstrate a first thickness towards a top and bottom surface and a second thickness towards a middle region between the top and bottom surfaces. In the same example, the first thickness is greater than the second thickness, thereby causing the implant to demonstrate an anisotropic stiffness (e.g., increased stiffness in a generally vertical, load bearing direction and reduced stiffness in a generally horizontal loading and translational axis).

According to one embodiment, by altering the thickness of the enhancement material, the magnitude of loads shared with PEEK material (to which the enhancement material is attached) is altered. In one or more embodiments, thicknesses of enhancement materials attached to an implant core body (e.g., a PEEK core body) are selected to optimize stress sharing between the enhancement material and the core body such that osteoblasts and their precursors at an implantation site respond as desired (e.g., in an osseointegration- and wound healing-favoring manner) to stress loading of the implant.

In various embodiments, the present implants include one or more of the enhancement material layers described herein (e.g., walls, sleeves, etc.) on perimeter surfaces and include one or more enhancement material spray layers on top and/or bottom surfaces. In at least one embodiment, the one or more enhancement material spray layers generally refer to layers of enhancement material (for example, titanium) that are sprayed onto surfaces of a core body (for example, perimeter, top, and bottom surfaces described herein). Thus, in one or more embodiments, the present enhancement material layer attachments for improving osseointegration are included in an implant in combination with one or more spray layers or coatings for improving osseointegration.

In at least one embodiment, one or more portions of a wall, inner sleeve, and/or outer sleeve described herein overlap and/or penetrate into a core body at a top and/or bottom surface thereof. According to one embodiment, the walls, inner sleeves, and/or outer sleeves are attached to the core body along the portions of the walls, inner sleeves, and/or outer sleeves that overlaps the top or bottom surfaces of the core body. In one example, a front wall 109 is attached to a core body 101. In the same example, the front wall 109 includes a top portion that passes through the top surface 127 and curves inwards towards a center 302 of the core body 101. In the example, the top portion of the front wall 109 is attached to the top surface of the core body 101.

In various embodiments, attachment of the walls, inner sleeves, and/or outer sleeves to the core body in a manner such that a portion thereof overlaps a top or bottom surface of the core body prevents bone growth in the in-contact surfaces of the core body and the corresponding wall, inner sleeve, and/or outer sleeve. In at least one embodiment, prevention of bone growth in between the core body and an attached enhancement material advantageously reduces a risk of displacement or detachment of the enhancement material.

### CONCLUSION

The embodiments were chosen and described in order to explain the principles of the claimed medical implants and methods of using the same and their practical application so as to enable others skilled in the art to utilize the medical implants and methods of using the same and various embodiments and with various modifications as are suited to the particular use contemplated. Alternative embodiments will become apparent to those skilled in the art to which the claimed medical implants and methods of using the same pertain without departing from their spirit and scope. Accordingly, the scope of the claimed medical implants and methods of using the same is defined by the appended claims rather than the foregoing description and the exemplary embodiments described therein.

What is claimed is:

1. A spinal implant comprising:
a PEEK body comprising:
a body top surface comprising a top surface outer edge;
a body bottom surface comprising a bottom surface outer edge; and
a perimeter surface between the top surface outer edge and the bottom surface outer edge and comprising four side surfaces; and
a titanium enhancement material layer that is about 0.001-0.010 inches thick and affixed to the PEEK body, and covers at least two of the four side surfaces of the perimeter surface, wherein the enhancement material layer comprises:
a layer top surface within the same plane as the body top surface;
a layer bottom surface within the same plane as the body bottom surface; and
at least one property modification.

2. The spinal implant of claim 1, wherein the at least one property modification comprises increased roughness of one or more surfaces of the enhancement material layer.

3. The spinal implant of claim 1, wherein the at least one property modification comprises increased porosity of one or more surfaces of the enhancement material layer.

4. The spinal implant of claim 1, wherein the enhancement material layer comprises one or more distinct portions, each distinct portion covering at least a portion of one of the four side surfaces of the perimeter surface.

5. The spinal implant of claim 4, wherein the enhancement material layer covers at least a portion of all four side surfaces of the perimeter surface.

6. The spinal implant of claim 1, wherein the enhancement material layer comprises a sleeve covering all four of the four side surfaces of the perimeter surface.

7. The spinal implant of claim 6, wherein:
the PEEK body defines at least one opening;
the body top surface comprises a top surface inner edge;
the body bottom surface comprises a bottom surface inner edge; and
the PEEK body comprises an inner perimeter surface between the top surface inner edge and the bottom surface inner edge.

8. The spinal implant of claim 7, wherein:
the spinal implant comprises a second enhancement material layer affixed to the PEEK body, covering at least a portion of the inner perimeter surface, and comprising:
a second layer top surface within the same plane as the body top surface; and
a second layer bottom surface with the same plane as the body bottom surface.

9. The spinal implant of claim 1, wherein the enhancement material layer defines one or more openings through the enhancement material layer.

10. The spinal implant of claim 9, wherein the one or more openings comprise encoded information.

11. The spinal implant of claim 10, wherein the encoded information is visible via a radiograph.

12. The spinal implant of claim 1, wherein the PEEK body comprises a shape selected from the group of: prisms, pyramids, solids of revolution.

13. A spinal implant comprising:
a PEEK body defining at least one opening and comprising:
a body top surface comprising a top surface outer edge;
a body bottom surface comprising a bottom surface outer edge; and
a perimeter surface between the top surface outer edge and the bottom surface outer edge; and
an enhancement material layer that is about 0.001-0.0394 inches thick and affixed to the PEEK body, and covers at least a portion of the perimeter surface, wherein the enhancement material layer comprises:
a layer top surface within the same plane as the body top surface;
a layer bottom surface within the same plane as the body bottom surface; and
at least one surface with increased roughness or porosity.

14. The spinal implant of claim 13, wherein the enhancement material layer comprises titanium.

15. The spinal implant of claim 14, wherein the perimeter surface comprises four side surfaces.

16. The spinal implant of claim 15, wherein the enhancement material layer comprises at least two portions, each portion covering one of the four side surfaces.

17. The spinal implant of claim 16, wherein the enhancement material layer comprises four portions, each portion covering one of the four side surfaces.

18. The spinal implant of claim 15, wherein the enhancement material layer comprises a sleeve covering all four of the four side surfaces.

19. The spinal implant of claim 18, wherein:
the body top surface comprises a top surface inner edge;
the body bottom surface comprises a bottom surface inner edge; and
the PEEK body comprises an inner perimeter surface between the top surface inner edge and the bottom surface inner edge.

20. The spinal implant of claim 17, wherein:
the spinal implant comprises a second enhancement material layer affixed to the PEEK body, covering at least a portion of the inner perimeter surface, and comprising:
a second layer top surface within the same plane as the body top surface; and
a second layer bottom surface with the same plane as the body bottom surface.
